(19) United States
(12) Patent Application Publication
(10) Pub. No.: US 2005/0197292 A1
(43) Pub. Date: Sep. 8, 2005

(54) Compositions and methods for treating T-cell mediated pathological conditions

(76) Inventors: Glennda Smithson, Guilford, CT (US); Mehdi Mesri, Gaithersburg, MD (US); Gary Starling, Clinton, CT (US)

Correspondence Address: Jenell Lawson;Intellectual Property, CuraGen Corporation, 555 Long Wharf Drive, New Haven, CT, 06551, US

(21) Appl. No.: 11/047,207

(22) Filed: Jan. 31, 2005

Related U.S. Application Data
(60) Provisional application No. 60/540,801, filed on Jan. 30, 2004.

(51) Int. Cl. A61K 38/17; C07K 14/72

(52) U.S. Cl. 514/12; 530/350

(57) ABSTRACT

The present invention relates to compositions and methods for preventing and/or treating inflammatory and autoimmune diseases. More particularly, the present invention relates to compositions comprising a CG57008 protein, fragment, derivative, variant, homolog, or analog thereof, and their uses in preventing and/or treating T-cell mediated diseases.

### RELATED APPLICATIONS

This application claims priority to U.S. Ser. No. 60/540,801 filed Jan. 30, 2004, U.S. Ser. No. 10/391,939, filed Mar. 19, 2003, U.S. Ser. No. 60/365,491 filed Mar. 19, 2002, and U.S. Ser. No. 60/410,618 filed Sep. 13, 2002, which is incorporated herein by reference in their entirety.

### FIELD OF THE INVENTION

The present invention relates to compositions and methods for preventing and/or treating inflammatory and autoimmune diseases. More particularly, the present invention relates to compositions comprising a CG57008 protein, fragment, derivative, variant, homolog, or analog thereof, and their uses in preventing and/or treating T-cell mediated diseases.

### BACKGROUND OF THE INVENTION

An optimal T cell response is achieved through signals delivered by the antigen-specific T cell receptor (TCR) and in combination with co-stimulatory or co-inhibitory signals (Lenschow et al. 1996, Annu Rev Immunol. 14: 233-58; Watts and DeBenedette 1999, Curr Opin Immunol. 11 (3): 286-93). Chronic antigenic stimulation results in the polarization of T helper cell subsets, namely TH1 and TH2 (Murphy et al, 2002). TH1 cells release interferon-gamma (IFN-gamma), interleukin 2 (IL-2) and tumor necrosis factor-alpha (TNF-alpha) whereas TH2 cells express IL-4, IL-5 and IL-13. Delayed-type hypersensitivity reactions (DTH) (Sher and Coffman, 1992, Annu Rev Immunol. 10: 385-409) and a variety of organ-specific autoimmune disorders including multiple sclerosis, inflammatory bowel disease, rheumatoid arthritis and type I diabetes mellitus are associated with aberrant TH1 responses (Kamradt and Mitchison, 2001, N Engl J Med 344, 655-664). In contrast, TH2 cell activation is essential in the pathogenesis of allergic asthma (Abbas et al., 1996, Nature 383, 787-793; Sher and Coffman, 1992). Much effort has gone into understanding the checkpoints that controls such responses, modulating the extent of T cell polarization and the overall degree of the T cell responses. Such investigated regulatory mechanisms include receptors such as cytotoxic T lymphocyte antigen 4 (CTLA4), cytokines such as IL-10 and regulatory cells CD4+ CD25+T cells.

A new family of genes encoding T cell, immunoglobulin domain and mucin domain (Tim) proteins (three in humans and eight in mice) have been described recently with emerging roles in immunity (Kuchroo et al., 2003, Nat Rev Immunol 3, 454-462; McIntire et al., 2001, Nat Immunol 2, 1109-1116). The Tim gene family members reside in chromosomal regions, 5q33.2 in human and 11B1.1 in mouse, and have been linked to allergy and autoimmune diseases (Shevach, 2002, Nat Rev Immunol 2, 389-400; Wills-Karp et al., 2003, Nat Immunol 4, 1050-1052). Hepatitis A virus cellular receptor (HAVcr-1) was originally discovered as a receptor for Hepatitis A virus (HAV) (Kaplan et al, 1996, EMBO J. 15(16): 4282-96), and was later cloned as kidney injury molecule 1 (KIM-1) (Ichimura et al 1998, J Biol Chem 273, 4135-4142; Han et al, 2002, Kidney Int 62, 237-244) and in mouse as Tim-1.

Kaplan et al U.S. Pat. No. 5,622,861 isolated the cellular receptor for hepatitis A virus from a cDNA library from a primary African Green Monkey Kidney (AGMK) cell line expressing the receptor. The utility of the polypeptides and nucleic acids was disclosed as to diagnose infection by hepatitis A virus, to separate hepatitis A virus from impurities in a sample, to treat infection as well as to prevent infection by hepatitis A virus. Furthermore, the polypeptides could be expressed in transformed cells and used to test efficacy of compounds in an anti-hepatitis A virus binding assay. The human homolog, hHAVcr-1, was described by Feigelstock et al, 1998 Journal of Virology 72(8): 6621-6628. The same molecules were described in WO 97/44460, WO98/53071 and U.S. Pat. No. 6,664,385 as Kidney Injury-related Molecules (KIM) that were found to be upregulated in renal tissue after injury to the kidney and the molecules were described as being useful in a variety of therapeutic interventions specifically renal disease, disorder or injury. WO02/098920 describes antibodies to KIM that are described as useful for inhibiting the shedding of KIM-1 polypeptide from KIM-1 expressing cells e.g. renal cells, or renal cancer cells.

Immunological interest in HAVcr-1 has grown since the mouse homologue Tim-1, was positionally cloned in a mouse model of allergic asthma, and was expressed by polarized TH2 cells. Interaction between HAV and Tim-1 (HAVcr-1) on CD4+ T cells may reduce TH2 differentiation and therefore reduce the development of asthma and allergy (McIntire et al., 2001). Such an interaction would also explain the inverse association of HAV infection with the development of asthma and allergy (Matricardi, 1997, British J Med 314, 999-1003; Matricardi et al., 1999, British J Med 314, 999-1003; Matricardi, 2000, British J Med 320, 412-417).

Tim-3, in contrast, was discovered for its TH1-specific expression (Monney et al., 2002, Nature 415, 536-541). In vivo administration of Tim-3 monoclonal antibodies in a TH1-mediated autoimmune disease, experimental allergic encephalomyelitis, resulted in more severe inflammatory events in the brain and exacerbated clinical disease. Furthermore, Tim-3 pathway blockade through treatment with a Tim-3-Ig fusion protein accelerated diabetes onset in nonobese diabetic (NOD) mice and abrogated the capacity of costimulatory blockade to induce tolerance. Therefore Tim-3 engagement by its putative ligand inhibits TH1-mediated inflammatory responses in vivo (Sanchez-Fueyo et al., 2003, Nat Immunol 4, 1093-1101). Tim-1 and Tim-3 thus are reciprocally expressed by TH2 and TH1 cells respectively. Ligands for both molecules remain to be identified.

T-cell mediated pathological conditions constitute major areas of unmet medical need. Thus, there is a need for therapeutic agents that regulate T cell activation and the concomitant production of cytokines for management and/or treatment of T-cell mediated pathological conditions.

### SUMMARY OF THE INVENTION

The present invention is based, in part, upon the discovery that CG57008 polypeptides inhibit the activation/proliferation of immune cells. In particular, it was determined that CG57008 inhibits the activation of T-cells and cytokine production from immune cells. Accordingly, the present invention provides compositions and methods for preventing and/or treating inflammatory and autoimmune diseases. More particularly, the present invention relates to compositions comprising CG57008 polypeptides, fragments, derivatives, variants, homologs, or analogs thereof for use in treating and/or preventing T-cell mediated inflammatory or immune disorders through inhibition of cytokine release and/or T-cell proliferation. The present invention also provides antibodies against a CG57008 protein.

In one aspect, the invention provides an isolated CG57008 protein. In some embodiments, the isolated protein comprises the amino acid sequence of SEQ ID NO:2. In other embodiments, the invention includes a variant of SEQ ID NO:2, in which some amino acids residues, e.g., no more than 1%, 2%, 3%, 5%, 10% or 15% of the amino acid sequences of SEQ ID NO;2 are changed. In some embodiments, the isolated CG57008 protein comprises the amino acid sequence of a mature form of an amino acid sequence given by SEQ ID NO:2 or a variant of a mature form of an amino acid sequence given by SEQ ID NO:2. Preferably, no more than 1%, 2%, 3%, 5%, 10% or 15% of the amino acid sequences of SEQ ID NO;2 are changed in the variant of the mature form of the amino acid sequence.

In another aspect, the invention provides a fragment of a CG57008 protein, including fragments of variant CG57008 proteins, mature CG57008 proteins, and variants of mature CG57008 proteins, as well as CG57008 proteins encoded by allelic variants and single nucleotide polymorphisms of CG57008 nucleic acids. An example of an CG57008 protein is a fragment that includes residues 1-283, 21-283, 21-359, 21-128 of CG57008 (SEQ ID NO:2).

In another aspect, the invention includes an isolated CG57008 nucleic acid molecule. The CG57008 nucleic acid molecule can include a sequence encoding any of the CG57008 proteins, variants, or fragments disclosed above, or a complement to any such nucleic acid sequence. In one embodiment, the sequences include those disclosed in SEQ ID NO:1, 3, 5, 6, 8, 9, 11, 13, 15, 17, 19, 21, 23, 25, 27, 29, 31, 33, 35, 37, 39, or 41, 43, 45, 47, 49, 51, 53, 55, 57, 59, 61, 63, 65, 67, 69, 71, 73, 75, 77, 79, and 81. In other embodiments, the CG57008 nucleic acids include a sequence wherein nucleotides different from those given in SEQ ID NO:1 may be incorporated. Preferably, no more than 1%, 2%, 3,%, 5%, 10%, 15%, or 20% of the nucleotides are so changed.

In one embodiment, the nucleic acid encodes a protein fragment that includes residues 21-283 of SEQ ID NO:2.

In other embodiments, the invention includes fragments or complements of these nucleic acid sequences. Vectors and cells incorporating CG57008 nucleic acids are also included in the invention. The present invention further provides methods of isolating a CG57008 protein by culturing the host cells containing a CG57008 nucleic acid in a suitable nutrient medium, and isolating one or more expressed CG57008 proteins.

The invention also includes antibodies that bind immunospecifically to any of the CG57008 proteins described herein. The CG57008 antibodies in various embodiments include, e.g., polyclonal antibodies, monoclonal antibodies, humanized antibodies and/or human antibodies.

The invention additionally provides pharmaceutical compositions that include a CG57008 protein, a CG57008 nucleic acid or a CG57008 antibody of the invention. Also included in the invention are kits that include, e.g., a CG57008 protein, a CG57008 nucleic acid or a CG57008 antibody.

Several methods are included in the invention. For example, a method is disclosed for inhibiting the activation and/or proliferation of immune cells. The method includes contacting immune cells with a composition comprising a CG57008 polypeptide. In some embodiments, the immune cells that are inhibited by CG57008 are T-cells, including helper T-cells (e.g., CD4+ or CD8+ T-cells), Th1 cells, or Th2 cells.

In another aspect, the invention provides a method of inhibiting cytokine release from immune cells (e.g., T-helper cells (including CD4+ T-cells and CD8+ T-cells) or T_{H}2 cells). The method includes contacting immune cells with a composition comprising a CG57008 polypeptide. The cytokine may be IFN-gamma, a member of the interleukin family (e.g., IL-2, IL-5, or IL-10), Tumor necrosis factor (TFN), or a combination thereof.

Also provided by the invention is a method of treating a pathological state in a subject, wherein the pathological state results from an improper immune response. The method includes administering to the subject a protein of the invention in an amount that is sufficient to alleviate the pathological state, wherein the CG57008 protein is a protein having an amino acid sequence at least 85%, 90%, 95%, 96%, 97%, 98%, or even 99% identical to a protein comprising an amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, and 82, or a biologically active fragment thereof. In another related method, an antibody of the invention is administered to the subject.

In one aspect, the invention provides a method of treating T-cell mediated diseases. The method includes administering to a subject a CG57008 protein of the invention in an amount and for a duration that is effective to inhibit T-cell activation and/or proliferation. As used herein, T-cells include, but are not limited to T-helper cells (e.g., CD4+ T-cells, CD8+ T-cells), Th2, and Th1 cells.

T-cell mediated diseases to be diagnosed, treated, prevented or delayed using the CG57008 nucleic acid molecules, proteins or antibodies include, but are not limited to atopic conditions (IgE-mediated allergic conditions), such as asthma, allergy, including allergic rhinitis, dermatitis, including psoriasis, pathogen susceptibilities, chronic inflammatory disease, organ-specific autoimmunity, including multiple sclerosis, Grave's disease, graft rejection, and graft-versus-host disease. Other immune disorders involving T-cell activation include, but are not limited to, Crohn's disease, systemic lupus erythematosus, reactive arthritis, rheumatoid arthritis, antigen-induced arthritis, insulin-dependent diabetes, contact dermatitis, gastrointestinal allergies, including food allergies, eosinophilia, nephritis, and certain viral infections, including HIV, and bacterial infections, including tuberculosis.

In a further aspect, the invention provides a method of treating cytokine-mediated diseases. The method includes administering to a subject a CG57008 protein of the invention in an amount and for a duration that is effective to inhibit cytokine release from immune cells (e.g., T-helper cells (including CD4+ T-cells and CD8+ T-cells) or T_{H}2 cells). In some embodiments, the cytokines to be inhibited by CG57008 include, but are not limited to IFN-gamma and interleukins (e.g., IL-2, IL-5, and IL-10).

In another aspect, the invention provides methods of treating inflammatory diseases/disorders. The method includes administering to a subject a CG57008 protein of the invention in an amount and for a duration that is effective to prevent and/or treat the inflammatory disease. In a specific embodiment, the inflammatory disease to be treated and/or prevented by the administration of a CG57008 protein is arthritis (such as, antigen-induced arthritis or rheumatoid arthritis). In another embodiment, the inflammatory disease to be prevented and/or treated is contact hypersensitivity or contact dermatitis.

In another aspect, the invention provides a method of diagnosing, treating, preventing or delaying an autoimmune disorder. The method includes administering to a subject, in need thereof, a CG57008 protein of the invention in an amount and for a duration that are effective to prevent and/or treat the autoimmune disease.

### Terminology

As used herein, the term “CG57008”, refers to a class of proteins (including peptides and polypeptides) or nucleic acids encoding such proteins or their complementary strands, where the proteins comprise an amino acid sequence of SEQ ID NO:2 (359 amino acids), or its fragments, derivatives, variants, homologs, or analogs. In a preferred embodiment, a CG57008 protein retains at least some biological activity of TIM-1. As used herein, the term “biological activity” means that a CG57008 protein possesses some but not necessarily all the same properties of (and not necessarily to the same degree as) TIM-1.

A member (e.g., a protein and/or a nucleic acid encoding the protein) of the CG57008 family may further be given an identification name. For example, CG57008-01 (SEQ ID NOs:1 and 2) represents TIM-1; CG57008-02 (SEQ ID NOs:3 and 4) represents a tagged version of the extracellular domain of TIM-1 without the signal sequence (aa 21-283); CG57008-06 (SEQ ID NOs:11 and 12) represents a single nucleotide polymorphism (“SNP”) of TIM-1 where one amino acid in CG57008-06 is different from SEQ ID NO:2 (the threonine at position 202 is changed to alanine, “²⁰²T→A”), which was shown herein to have immunosuppressant properties. Table 1 shows a summary of some of the CG57008 family members. In one embodiment, the invention includes a variant of TIM-1 protein, in which some amino acids residues, e.g., no more than 1%, 2%, 3%, 5%, 10% or 15% of the amino acid sequence of TIM-1 (SEQ ID NO:2), are changed. In another embodiment, the invention includes nucleic acid molecules that can hybridize to TIM-1 under stringent hybridization conditions.

TABLE 1
Summary of some of the CG57008 family members

| | SEQ ID | SEQ | | |
|---|---|---|---|---|
| | NO | ID NO | Corresponding | |
| Internal | (nucleic | (amino | amino acids of | |
| Identification | acid) | acid) | CG57008-01 | Description |
| CG57008-01 | 1 | 2 | 1-359 | HuHAVcr-1; |
| | | | | WO200298920 KIM-1 SEQ ID |
| | | | | 8; |
| | | | | WO2003002722 huTIM-1 |
| | | | | allele 1 |
| CG57008-02 | 3 | 4 | 21-283 | ECD, V5, His |
| CG57008-03 | 5 | 6 | 21-359 | mature |
| CG57008-04 | 7 | 8 | 21-128 | Ig domain |
| CG57008-05 | 9 | 10 | 21-282 | IgK, ECD*, Fc, V5, His |
| CG57008-06 | 11 | 12 | 21-283 (T202A) | ECD-SNP |
| CG57008-07 | 13 | 14 | TGST-(1-359)-VDG | Full length plus cloning vector |
| | | | | restriction sites |
| CG57008-08 | 15 | 16 | (1-359)-VDG | Full length plus one cloning |
| | | | | vector restriction site |
| CG57008-09 | 17 | 18 | 1-283-Fc | signal, ECD, Fc |
| CG57008-10 | 19 | 20 | (21-154)(insert | ECD 6aa insertion 1aa |
| | | | TVPMT)(155-192) | deletion |
| | | | (delete 193)(194-283) | |
| CG57008-11 | 21 | 22 | IgK (21- | Igkappa, ECD SNP, Fc, V5, |
| | | | 283, T202A)FcV5His | His |
| CG57008-12 | 23 | 24 | IgK mature(21- | Mature Igkappa, ECD SNP, |
| | | | 283, T202A)FcV5His | Fc, V5, His |
| CG57008-13 | 25 | 26 | IgK(21-129)FcV5His | Igkappa, Ig domain, Fc, V5, |
| | | | | His |
| CG57008-14 | 27 | 28 | IgK mature (21-129 | Igkappa mature, Ig domain, |
| | | | )FC V5 His | Fc, V5, His |
| CG57008-15 | 29 | 30 | IgK mature (21-283, | Igkappa mature, ECD SNP, |
| | | | T202A) V5 His | V5, His |
| CG57008-16 | 31 | 32 | IgK (21-283, | Igkappa ECD SNP, V5, His |
| | | | T202A)V5His | |
| SNP | 33 | 34 | 1-359, T202A | Human TIM-1 - SNP T202A |
| 13378265 of | | | | |
| CG57008-01 | | | | |
| SNP | 35 | 36 | 1-359, P174L | TIM-1 - SNP P174L (Homo |
| 13378266 of | | | | sapiens) |
| CG57008-01 | | | | |
| SNP | 37 | 38 | 1-359, V138I | TIM-1 - SNP V138I (Homo |
| 13378615 of | | | | sapiens) |
| CG57008-01 | | | | |
| SNP | 39 | 40 | 1-359, S51L | TIM-1 - SNP S51L (Homo |
| 13382206 of | | | | sapiens) |
| CG57008-01 | | | | |
| SNP | 41 | 42 | 1-359, A96V | TIM-1 - SNP A96V (Homo |
| 13382207 of | | | | sapiens) |
| CG57008-01 | | | | |
| CG201877- | 43 | 44 | | Tim1 - Mus musculus; Mature |
| 02 (ECD/FC) | | | | Igkappa, ECD, Fc, V5, His |
| CG51117-05 | 45 | 46 | | Nephronectin - Homo sapiens |
| (mature) | | | | |
| CG51373-07 | 47 | 48 | | Nephrin - Homo sapiens |
| (mature) | | | | |
| CG57008-17 | 49 | 50 | | ForSall-RevNotl |
| CG57008-18 | 51 | 52 | 1-273 | Signal, ECD |
| CG57008-19 | 53 | 54 | | 57008-07 SDMThrtoALa |
| CG57008-20 | 55 | 56 | | F1 (Nhel 1-)-R1 (263 Xhol) |
| CG57008-21 | 57 | 58 | | F1 (21Nhe-)-R1 (515 EcoRl) |
| CG57008-22 | 59 | 60 | IgK, 21-283 (T202A), | Plasmid#4096 SDM: T202A |
| | | | Fc | |
| CG57008-23 | 61 | 62 | | ECD with IgGkappa, Fc |
| CG57008-24 | 63 | 64 | | Native secretion signal, ECD |
| | | | | with deletion 9, 3′, Fc tagged |
| CG57008-25 | 65 | 66 | | Native secretion signal, ECD |
| | | | | with deletion #3, 3′, Fc tagged |
| CG57008-26 | 67 | 68 | | Native secretion signal, ECD |
| | | | | with deletion #8, 3′, Fc tagged |
| CG57008-27 | 69 | 70 | | Native secretion signal, ECD |
| | | | | with deletion #6, 3′, Fc tagged |
| CG57008-28 | 71 | 72 | | Native secretion signal, ECD |
| | | | | with deletion #4, 3′, Fc tagged |
| CG57008-29 | 73 | 74 | | Native secretion signal, ECD |
| | | | | with deletion #5, 3′, Fc tagged |
| CG57008-30 | 75 | 76 | | Native secretion signal, ECD |
| | | | | with deletion #10, 3′, Fc |
| | | | | tagged |
| CG57008-31 | 77 | 78 | | Native secretion signal, ECD |
| | | | | with deletion #1, 3′, Fc tagged |
| CG57008-32 | 79 | 80 | | Native secretion signal, ECD |
| | | | | with deletion #7, 3′, Fc tagged |
| CG57008-33 | 81 | 82 | | Native secretion signal, ECD |
| | | | | with deletion #2, 3′, Fc tagged |

As used herein, the term “effective amount” refers to the amount of a therapy (e.g., a composition comprising a CG57008 protein) which is sufficient to reduce and/or ameliorate the severity and/or duration of a T-cell mediated or cytokine-mediated disorder, or one or more symptoms thereof, prevent the advancement of a disease, cause regression of a disease, prevent the recurrence, development, or onset of one or more symptoms associated with a disease, or enhance or improve the prophylactic or therapeutic effect(s) of another therapy.

As used herein, the term “TIM-1” refers to a protein comprising an amino acid sequence of SEQ ID NO:2, or a nucleic acid sequence encoding such a protein or the complementary strand thereof.

As used herein, the term “hybridizes under stringent conditions” describes conditions for hybridization and washing under which nucleotide sequences at least 30% (preferably, 35%, 40%, 45%, 50%, 55%, 60%, 65%, 70%, 75%, 80%, 85%, 90%, 95% or 98%) identical to each other typically remain hybridized to each other. Such stringent conditions are known to those skilled in the art and can be found in Current Protocols in Molecular Biology, John Wiley & Sons, N.Y. (1989), 6.3.1-6.3.6. In one, non limiting example, stringent hybridization conditions comprise a salt concentration from about 0.1 M to about 1.0 M sodium ion, a pH from about 7.0 to about 8.3, a temperature is at least about 60° C., and at least one wash in 0.2×SSC, 0.01% BSA. In another non-limiting example, stringent hybridization conditions are hybridization at 6× sodium chloride/sodium citrate (SSC) at about 45° C., followed by one or more washes in 0.1×SSC, 0.2% SDS at about 68° C. In yet another non-limiting example, stringent hybridization conditions are hybridization in 6×SSC at about 45° C., followed by one or more washes in 0.2×SSC, 0.1% SDS at 50-65° C. (i.e., one or more washes at 50° C., 55° C., 60° C. or 65° C.). It is understood that the nucleic acids of the invention do not include nucleic acid molecules that hybridize under these conditions solely to a nucleotide sequence consisting of only A or T nucleotides.

As used herein, the term “isolated” in the context of a protein agent refers to a protein agent that is substantially free of cellular material or contaminating proteins from the cell or tissue source from which it is derived, or substantially free of chemical precursors or other chemicals when chemically synthesized. The language “substantially free of cellular material” includes preparations of a protein agent in which the protein agent is separated from cellular components of the cells from which it is isolated or recombinantly produced. Thus, a protein agent that is substantially free of cellular material includes preparations of a protein agent having less than about 30%, 20%, 10%, or 5% (by dry weight) of host cell proteins (also referred to as a “contaminating proteins”). When the protein agent is recombinantly produced, it is also preferably substantially free of culture med medium, i.e., culture medium represents less than about 20%, 10%, or 5% of the volume of the protein agent preparation. When the protein agent is produced by chemical synthesis, it is preferably substantially free of chemical precursors or other chemicals, i.e., it is separated from chemical precursors or other chemicals that are involved in the synthesis of the protein agent. Accordingly, such preparations of a protein agent have less than about 30%, 20%, 10%, 5% (by dry weight) of chemical precursors or compounds other than the protein agent of interest. In a specific embodiment, protein agents disclosed herein are isolated.

As used herein, the term “isolated” in the context of nucleic acid molecules refers to a nucleic acid molecule that is separated from other nucleic acid molecules that are present in the natural source of the nucleic acid molecule. Moreover, an “isolated” nucleic acid molecule, such as a cDNA molecule, can be substantially free of other cellular material or culture medium when produced by recombinant techniques, or substantially free of chemical precursors or other chemicals when chemically synthesized. In a specific embodiment, nucleic acid molecules are isolated.

As used herein, the terms “prevent,” “preventing,” and “prevention” refer to the prevention of the recurrence, onset, or development of a T-cell mediated or cytokine-mediated disorder or one or more symptoms thereof in a subject resulting from the administration of a therapy (e.g., a composition comprising a CG57008 protein), or the administration of a combination of therapies.

As used herein, the terms “subject” and “subjects” refer to an animal, preferably a mammal, including a non-primate (e.g., a cow, pig, horse, cat, or dog), a primate (e.g., a monkey, chimpanzee, or human), and more preferably a human. The term “subject” is used interchangeably with “patient” in the present invention.

As used herein, the terms “treat,” “treatment,” and “treating” refer to the reduction of the progression, severity, and/or duration of a T-cell mediated and/or cytokine-mediated disorder or amelioration of one or more symptoms thereof, wherein such reduction and/or amelioration result from the administration of one or more therapies (e.g., a composition comprising a CG57008 protein).

As used herein, the term “therapeutically effective amount” refers to the amount of a therapy (e.g., a composition comprising a CG57008 protein), which is sufficient to reduce the severity of a T-cell mediated or cytokine-mediated disorder, reduce the duration of a disease, prevent the advancement of a disease, cause regression of a disease, ameliorate one or more symptoms associated with a disease, or enhance or improve the therapeutic effect(s) of another therapy.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DESCRIPTION OF THE DRAWINGS

FIG. 1 depicts the structure of some CG57008 proteins

FIG. 2 (a-e) depict the inhibition of TCR-mediated responses by CG57008. These data are representative of at least three independent experiments.

FIG. 2(a) depicts the inhibition of IFN-gamma release by CG57008. Purified CD4+ T cells from healthy volunteers were stimulated with immobilized anti-CD3 and two concentrations of CG57008-15 protein or a control protein. Aliquots of supernatants were collected at 72 h after initiation of cultures and IFN-gamma measured by ELISA.

FIG. 2(b) depicts the inhibition of IL-5 release by CG57008. Purified CD4+ T cells from healthy volunteers were stimulated with immobilized anti-CD3 and two concentrations of CG57008-15 protein or a control protein. Aliquots of supernatants were collected at 72 h after initiation of cultures and IL-5 measured by ELISA.

FIG. 2(c) depicts the inhibition of proliferation by CG57008. Purified CD4+ T cells from healthy volunteers were stimulated with immobilized anti-CD3 and two concentrations of CG57008-15 protein or a control protein. Proliferation was measured by BrdU incorporation in triplicate after 72 h.

FIG. 2(d) depicts the inhibition of IFN-gamma release by CG57008. Purified CD4+ T cells from healthy volunteers were stimulated by increasing concentrations of immobilized anti-CD3 and a single concentration of CG57008 proteins or control proteins for 72 h. IFN-gamma was measured in culture supernatants by ELISA.

FIG. 2(e) depicts the inhibition of IL-5 release by CG57008. Purified CD4+ T cells from healthy volunteers were stimulated by increasing concentrations of immobilized anti-CD3 and a single concentration of CG57008 proteins or control proteins for 72 h. IL-5 was measured in culture supernatants by ELISA.

FIGS. 3(a-n) depict the inhibition of TCR-CD28 co-stimulatory responses by CG57008. These data are representative of at least three independent experiments.

FIG. 3(a) depicts the inhibition of IFN-gamma release by CG57008. 96-well plates were coated with the anti-CD3 and anti-CD28 mAb at 150 ng/ml and 1 μg/ml respectively in PBS overnight at 4° C., aspirated and coated with various concentrations of the indicated recombinant CG57008 protein variants or a control protein for 4 hr at 37° C. Wells were aspirated and plated with 150 μl of purified Human CD8+ T cells at a concentration of 0.7×10⁶ cells/ml for 72 h at 37° C. Supernatants were harvested at 72 h for IFN-gamma ELISA.

FIG. 3(b) depicts the inhibition of IL-2 release by CG57008. Cells were processed as described for FIG. 3(a). In addition, some cells were treated with mouse CG201877 protein instead of human CG57008. Supernatants were harvested at 72 h for IL-2 ELISA.

FIG. 3(c) depicts the inhibition of IL-5 release by CG57008. Cells were processed as described for FIG. 3(b). Supernatants were harvested at 72 h for IL-5 ELISA.

FIG. 3(d) depicts the inhibition of IL-10 release by CG57008. Cells were processed as described for FIG. 3(b). Supernatants were harvested at 72 h for IL-10 ELISA.

FIG. 3(e) depicts the inhibition of proliferation by CG57008. Cells were processed as described for FIG. 3(b). In addition, cells were pulsed with 0.5 μCi of [³H] thymidine 8 hr prior to harvesting, and incorporation of radioactivity was measured at 72 hours.

FIG. 3(f) depicts the inhibition of IFN-gamma release by CG57008. Cells were processed as described for FIG. 3(b), except human CD4+ T cells were used. Supernatants were harvested at 72 h for IFN-gamma ELISA.

FIG. 3(g) depicts the inhibition of IL-2 release by CG57008. Cells were processed as described for FIG. 3(f). Supernatants were harvested at 72 h for IL-2 ELISA.

FIG. 3(h) depicts the inhibition of IL-5 release by CG57008. Cells were processed as described for FIG. 3(f). Supernatants were harvested at 72 h for IL-5 ELISA.

FIG. 3(i) depicts the inhibition of IL-10 release by CG57008. Cells were processed as described for FIG. 3(f). Supernatants were harvested at 72 h for IL-10 ELISA.

FIG. 3(j) depicts the inhibition of proliferation by CG57008. Cells were processed as described for FIG. 3(e), except human CD4+ T cells were used.

FIG. 3(k) depicts the inhibition of IFN-gamma release by CG57008. Cells were processed as described for FIG. 3(a), except polarized T_{H}2 cells were used instead of CD8+ cells.

FIG. 3(l) depicts the inhibition of IL-5 release by CG57008. Cells were processed as described for FIG. 3(k). Supernatants were harvested at 72 h for IL-5 ELISA.

FIG. 3(m) depicts the inhibition of IFN-gamma release by CG57008. Cells were processed as described for FIG. 3(a), except mouse CD8+ cells were used instead of human CD8+ cells.

FIG. 3(n) depicts the inhibition of IFN-gamma release by CG57008. Cells were processed as described for FIG. 3(m), except mouse CD4+ cells were used instead mouse CD8+ cells.

FIG. 4 depicts the change in ear swelling in response to CG57008 treatment in an Oxazalone-Induced Mouse Model of Contact Hypersensitivity. Animals were treated on day—1, 0, and 2 with variant CG57008 proteins, or control reagents. Data shows the change in ear swelling based on 10 animals at days 6-7 and 7 animals for the remainder of the timepoints.

FIG. 5 depicts the change in leukocyte infiltration in response to CG57008-05 protein treatment in an Oxazalone-Induced Mouse Model of Contact Hypersensitivity. Ears were fixed, embedded in paraffin, and stained with hematoxylin and eosin. The level of leukocyte infiltration was scored from none (0) to severe (5) in the treated ears (left) of 3 animals from each group and the untreated ears (right) of the PBS controls. Leukocytes were scored as polymorphonuclear cells (PMN)The percentages shown are the percentage of appropriate control (PBS for cyclosporine and CG57008-15; IgG1 for CG57008-05).

FIG. 6(a) depicts the effect of CG57008-05 treatment, at the time of sensitization, on soft tissue swelling (ankle width).

FIG. 6(b) depicts the effect of CG57008-05 treatment, at the time of sensitization, on histopathological inflammation (scoring 1-3).

FIG. 7(a) depicts the effect of CG57008-05 treatment, at the time of challenge, on soft tissue swelling (ankle width).

FIG. 7(b) depicts the effect of CG57008-05 treatment, at the time of challenge, on histopathological inflammation (scoring 1-3).

FIG. 8(a) depicts the effect of CG57008-05 treatment, at the time of challenge, on histopathological inflammation in the knee joint (scoring 1-5).

FIG. 8(b) depicts the effect of CG57008-05 treatment, at the time of challenge, on pannus formation, cartilage degradation and bone lesions in the knee joint (scoring 1-5).

FIG. 9(a) depicts the effect of CG57008-15 treatment, at the time of challenge, on soft tissue swelling (ankle width).

FIG. 9(b) depicts the effect of CG57008-22 treatment, at the time of challenge, on soft tissue swelling (ankle width).

FIG. 10 depicts sequence data for the CG57008 isoforms listed in Table 1.

### DETAILED DESCRIPTION OF THE INVENTION

The present invention is based, in part, upon the discovery that CG57008 polypeptides inhibit T-cell activation/proliferation and/or cytokine production. Accordingly, the present invention provides composition and methods for preventing and/or treating T-cell mediated and cytokine mediated disorders.

For clarity of disclosure, and not by way of limitation, the detailed description of the invention is divided into the following subsections:
(i) CG57008
(ii) Methods of Preparing CG57008
(iii) Antibodies to CG57008
(iv) Characterization and Demonstration of CG57008 Activities and Monitoring Effects During Treatment
(v) Uses of CG57008
(vi) Administration, Pharmaceutical Compositions, and Kits

### 5.1 CG57008

The present invention provides for compositions comprising CG57008 for prevention and/or treatment of T-cell mediated and cytokine mediated disorders (e.g., including inflammatory diseases, such as arthritis, contact hypersensitivity, and contact dermatitis). As used herein, the term “CG57008” refers to a class of proteins (including peptides and polypeptides) or nucleic acids encoding such proteins or their complementary strands, where the proteins comprise an amino acid sequence of SEQ ID NO:2, or its fragments, derivatives, variants, homologs, or analogs. More specifically, the precursor form or preprotein exemplified by CG57008-01 (SEQ ID NO.2) is the full length gene product, encoded by the open reading frame ORF of the nucleic acid SEQ ID NO.1. The mature polypeptide exemplified by CG57008-03, SEQ ID NO.6 (corresponding to amino acid residues 21-359 of CG57008-01, SEQ ID NO.2) encoded by nucleotide SEQ ID NO.5 arises, for example as a result of one or more naturally occurring processing steps that may take place within the cell in which the gene product arises. The mature form may arise as a result of cleavage of the N-terminal methionine residue or N-terminal signal sequence, or post-translational modification such as glycosylation, myristylation or phosphorylation. The extracellular domain (ECD) CG57008-O₂, SEQ ID NO.4 (corresponding to amino acid residues 21 to 283 of CG57008-01, SEQ ID NO.2), encoded by nucleotide SEQ ID NO.3 can be used in the method of the invention. It has been found that the Ig domain CG57008-04, SEQ ID NO.8 (corresponding to amino acid residues 21-128 of CG57008-01, SEQ ID NO. 2), is effective as an immunosuppressant and can be used in the method of the invention.

In one embodiment, a CG57008 protein is a variant of TIM-1. It will be appreciated by those skilled in the art that DNA sequence polymorphisms that lead to changes in the amino acid sequences of the TIM-1 protein may exist within a population (e.g., the human population). Such genetic polymorphism in the TIM-1 gene may exist among individuals within a population due to natural allelic variation. Such natural allelic variations can typically result in 1-5% variance in the nucleotide sequence of the TIM-1 gene. Any and all such nucleotide variations and resulting amino acid polymorphisms in the TIM-1 protein, which are the result of natural allelic variation of the TIM-1 protein, are intended to be within the scope of the invention.

In one embodiment, a CG57008 is CG57008-06 (SEQ ID NOs:11 and 12), which is a single nucleotide polymorphism (“SNP”) of TIM-1 (i.e., ²⁰² T→A). Variants containing the ²⁰²T→A SNP (i.e., CG57008-22) were found herein to have immunosuppressant properties (see, e.g., Example 5, infra).

Examples of other such variants include but are not limited to those shown in Table 2. Nucleotide positions correspond that position in the nucleotide sequence of CG57008-01, SEQ ID NO. 1. Amino acid position corresponds to that position in the amino acid sequence of CG57008-01, SEQ ID NO. 2.

TABLE 2
Polymorphic Variants of CG57008-01

| | | | | SEQ | | | | SEQ |
|---|---|---|---|---|---|---|---|---|
| | Nucleotides | | | ID | Amino Acids | | | ID |
| Variant | Position | Initial | Modified | NO. | Position | Initial | Modified | NO. |
| SNP 13382206 | 203 | C | T | 39 | 51 | Ser | Leu | 40 |
| SNP 13382207 | 338 | C | T | 41 | 96 | Ala | Val | 42 |
| SNP 13378615 | 463 | G | A | 37 | 138 | Val | Ile | 38 |
| SNP 13378266 | 572 | C | T | 35 | 174 | Pro | Leu | 36 |
| SNP 13378265 | 655 | A | G | 33 | 202 | Thr | Ala | 34 |

In another embodiment, CG57008 refers to a nucleic acid molecule encoding a TIM-1 protein from other species or the protein encoded thereby, and thus has a nucleotide or amino acid sequence that differs from the human sequence of TIM-1. Nucleic acid molecules corresponding to natural allelic variants and homologues of the TIM-1 cDNAs of the invention can be isolated based on their homology to the human TIM-1 nucleic acids disclosed herein using the human cDNAs, or a portion thereof, as a hybridization probe according to standard hybridization techniques under stringent hybridization conditions.

In another embodiment, CG57008 refers to a fragment of a TIM-1 protein, including fragments of variant TIM-1 proteins, mature TIM-1 proteins (e.g., CG57008-03), and variants of mature TIM-1 proteins, as well as TIM-1 proteins encoded by allelic variants and single nucleotide polymorphisms of TIM-1 nucleic acids (e.g., CG57008-06). Examples of TIM-1 protein fragments found herein to have immunosuppressant properties include, but are not limited to, fragments consisting of the following amino acid residues: 1-283, 21-283, 21-359, 21-128 of TIM-1 (SEQ ID NO:2). In one embodiment, CG57008 refers to a nucleic acid encodes a protein fragment that includes residues 1-283, 21-283, 21-359, 21-128 of TIM-1 (SEQ ID NO:2).

The invention also encompasses derivatives and analogs of TIM-1. The production and use of derivatives and analogs related to TIM-1 are within the scope of the present invention.

In a specific embodiment, the derivative or analog is functionally active, i.e., capable of exhibiting one or more functional activities associated with a full-length, wild-type TIM-1. Derivatives or analogs of TIM-1 can be tested for the desired activity by procedures known in the art, including but not limited to, using appropriate cell lines, animal models, and clinical trials.

In particular, TIM-1 derivatives can be made via altering TIM-1 sequences by substitutions, insertions or deletions that provide for functionally equivalent molecules. In one embodiment, such alteration of an TIM-1 sequence is done in a region that is not conserved in the TIM protein family. Due to the degeneracy of nucleotide coding sequences, other DNA sequences which encode substantially the same amino acid sequence as TIM-1 may be used in the practice of the present invention. These include, but are not limited to, nucleic acid sequences comprising all or portions of TIM-1 that are altered by the substitution of different codons that encode a functionally equivalent amino acid residue within the sequence, thus producing a silent change. Likewise, the TIM-1 derivatives of the invention include, but are not limited to, those containing, as a primary amino acid sequence, all or part of the amino acid sequence of TIM-1 including altered sequences in which functionally equivalent amino acid residues are substituted for residues within the sequence resulting in a silent change. For example, one or more amino acid residues within the sequence can be substituted by another amino acid of a similar polarity that acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. TIM-1 derivatives of the invention also include, but are not limited to, those containing, as a primary amino acid sequence, all or part of the amino acid sequence of TIM-1 including altered sequences in which amino acid residues are substituted for residues with similar chemical properties. In a specific embodiment, 1, 2, 3, 4, or 5 amino acids are substituted.

Derivatives or analogs of TIM-1 include, but are not limited to, those proteins which are substantially homologous to TIM-1 or fragments thereof, or whose encoding nucleic acid is capable of hybridizing to the TIM-1 nucleic acid sequence.

In a specific embodiment, chimeric or fusion proteins including CG57008 polypeptides may be used in the method of the invention. As used herein, a “chimeric protein” or “fusion protein” comprises a CG57008 polypeptide operatively-linked to a non-CG57008 polypeptide. Within such a fusion protein, the CG57008 polypeptide can correspond to all or a portion of a CG57008 protein. In one embodiment, a CG57008 fusion protein comprises at least one biologically-active portion of a CG57008 protein. Within the fusion protein, the CG57008 polypeptide and the non-CG57008 polypeptide are “operatively-linked”, that is they are fused in-frame with one another. The non-CG57008 polypeptide can be fused to the N-terminus or C-terminus of the CG57008 polypeptide. For example, the fusion protein may be a CG57008 protein containing a heterologous signal sequence at its N-terminus (e.g., CG57008-11, CG57008-12, and CG57008-15). In certain host cells (e.g., mammalian host cells), expression and/or secretion of CG57008 can be increased through use of a heterologous signal sequence. In yet another example, the fusion protein is a CG57008-immunoglobulin fusion protein in which the CG57008 sequences are fused to sequences derived from a member of the immunoglobulin protein family. The CG57008-immunoglobulin fusion proteins can be incorporated into pharmaceutical compositions and administered to a subject to inhibit an immunological response according to the present invention.

A CG57008 chimeric or fusion protein for use in the method of the invention may be chemically modified for the purpose of improving bioavailability, and increasing efficacy, solubility and stability. For example, the protein may be covalently or non-covalently linked to polyethylene glycol (PEG).

A CG57008 chimeric or fusion protein for use in the method of the invention can be produced by standard recombinant DNA techniques. For example, DNA fragments coding for the different polypeptide sequences may be ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. Furthermore, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence [see, e.g., Ausubel, et al. (eds.) CURRENT PROTOCOLS IN MOLECULAR BIOLOGY, John Wiley & Sons, (1992)]. Moreover, many expression vectors are commercially available that already encode a fusion moiety (e.g., a GST polypeptide). A CG57008-encoding nucleic acid can be cloned into such an expression vector such that the fusion moiety is linked in-frame to the CG57008 protein. The fusion protein can be a CG57008 protein fused to a His tag or epitope tag (e.g. V5) to aid in the purification and detection of the recombinant CG57008 protein, or to mask the immune response in a subject.

In some embodiments, a CG57008 protein can be modified so that it has an extended half-life in vivo using any methods known in the art. For example, Fc fragment of human IgG or inert polymer molecules such as high molecular weight polyethyleneglycol (PEG) can be attached to a CG57008 protein with or without a multifunctional linker either through site-specific conjugation of the PEG to the N- or C-terminus of the protein or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the CG57008 protein. Unreacted PEG can be separated from CG57008-PEG conjugates by size-exclusion or by ion-exchange chromatography. PEG-derivatized conjugates can be tested for in vivo efficacy using methods known to those of skill in the art.

In a preferred embodiment, a fusion protein for use in the present invention is CG57008-22.

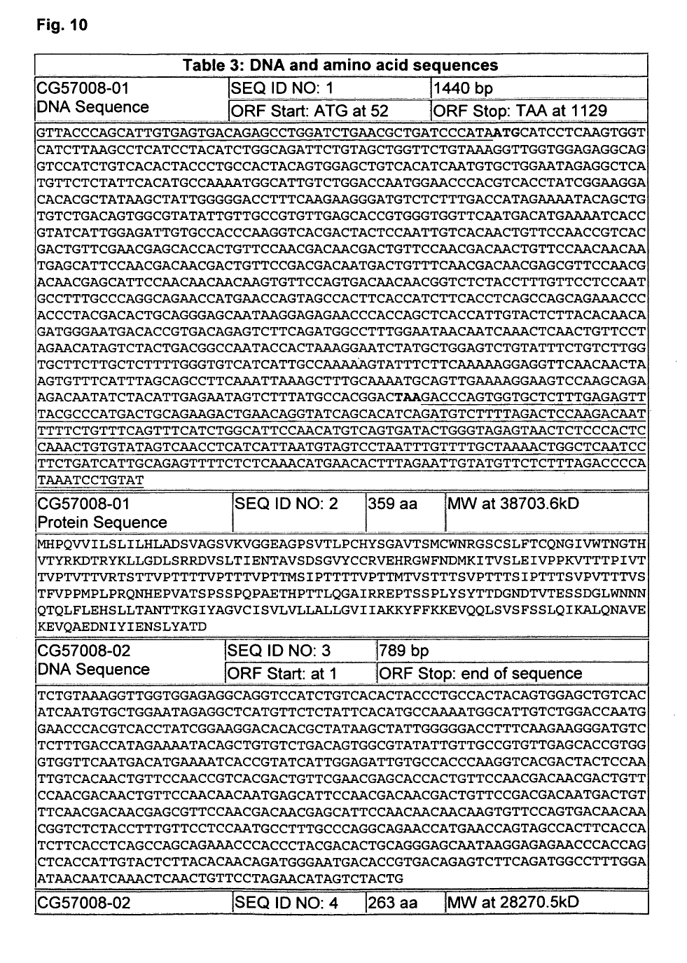

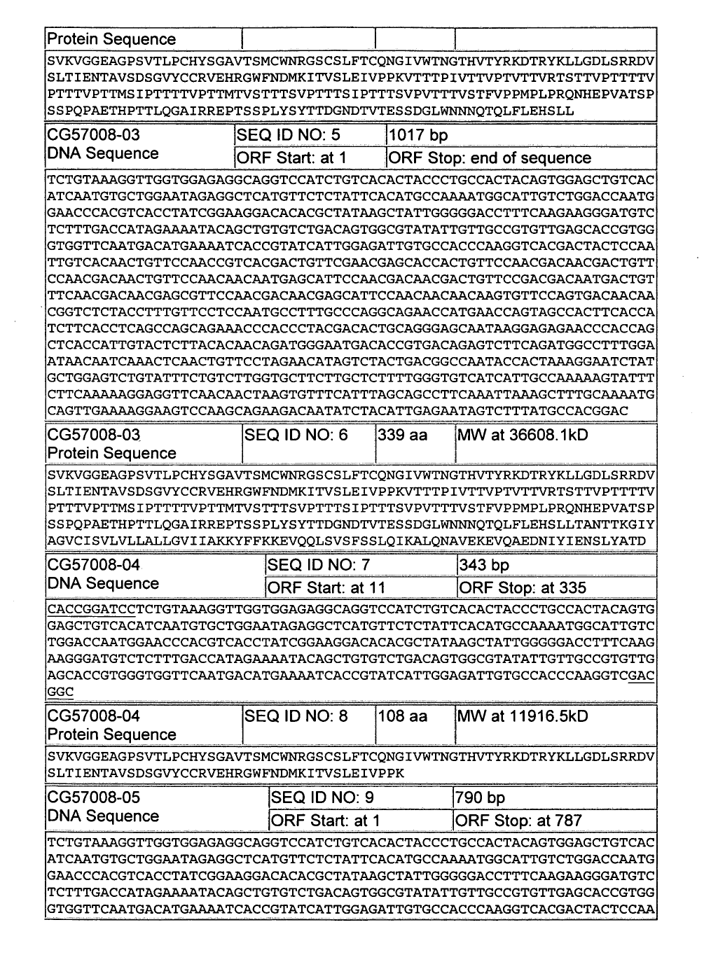

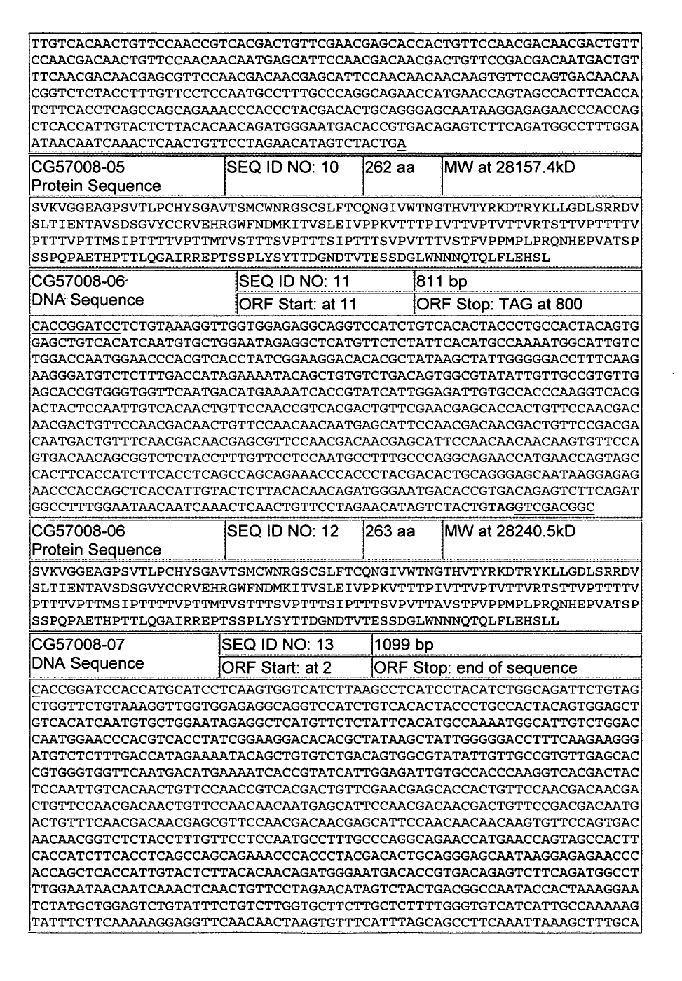

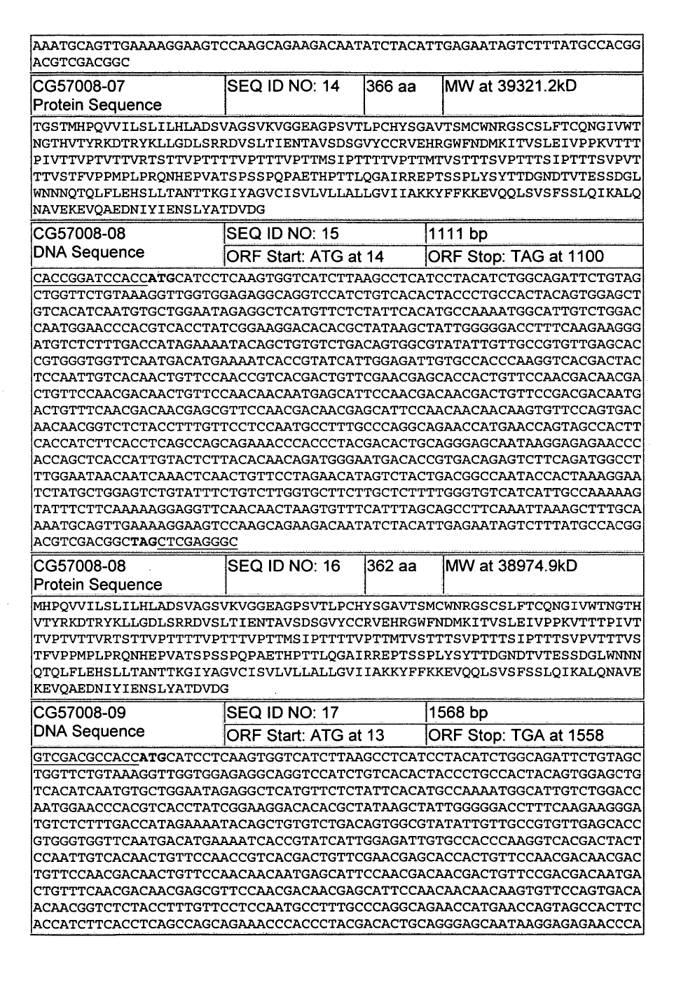

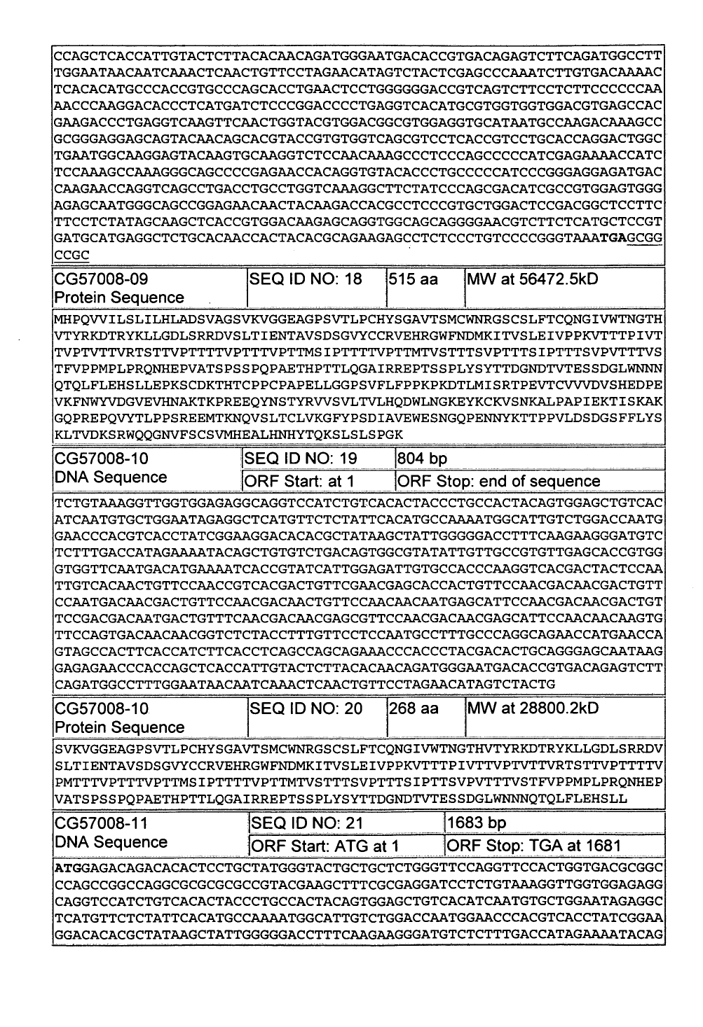

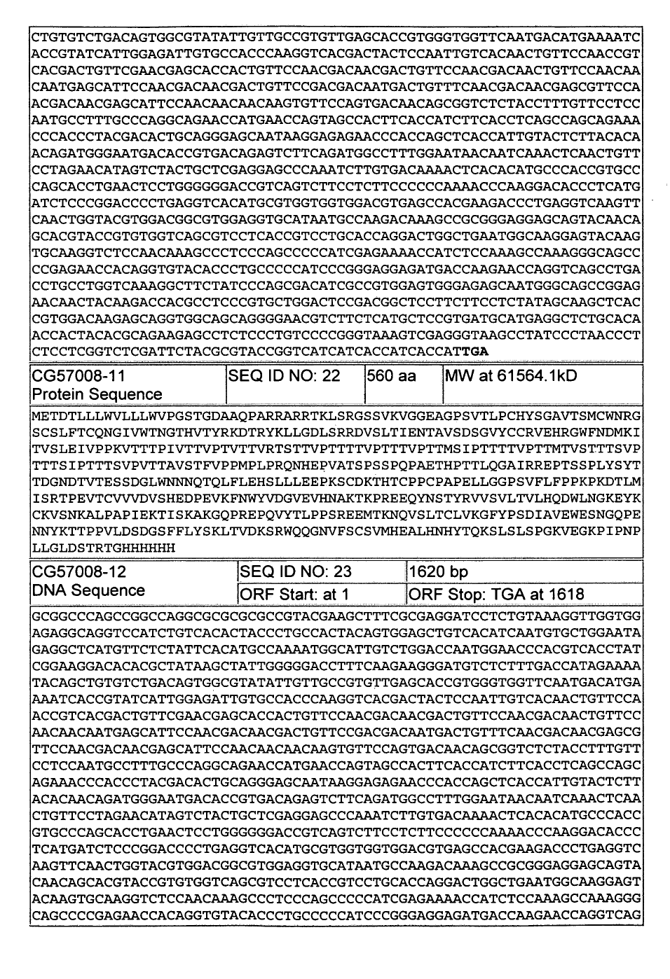

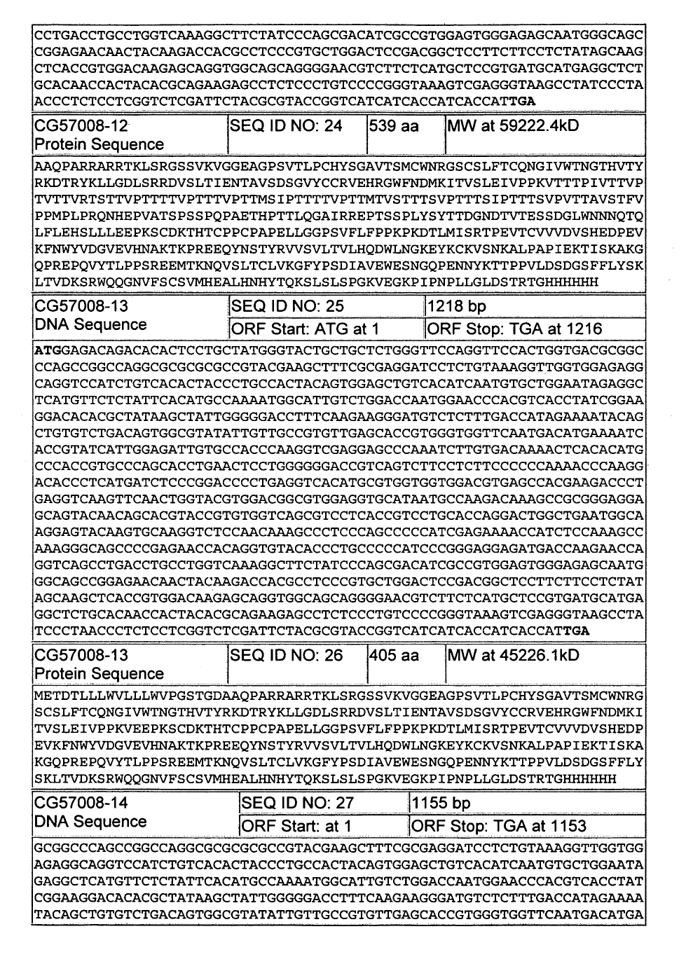

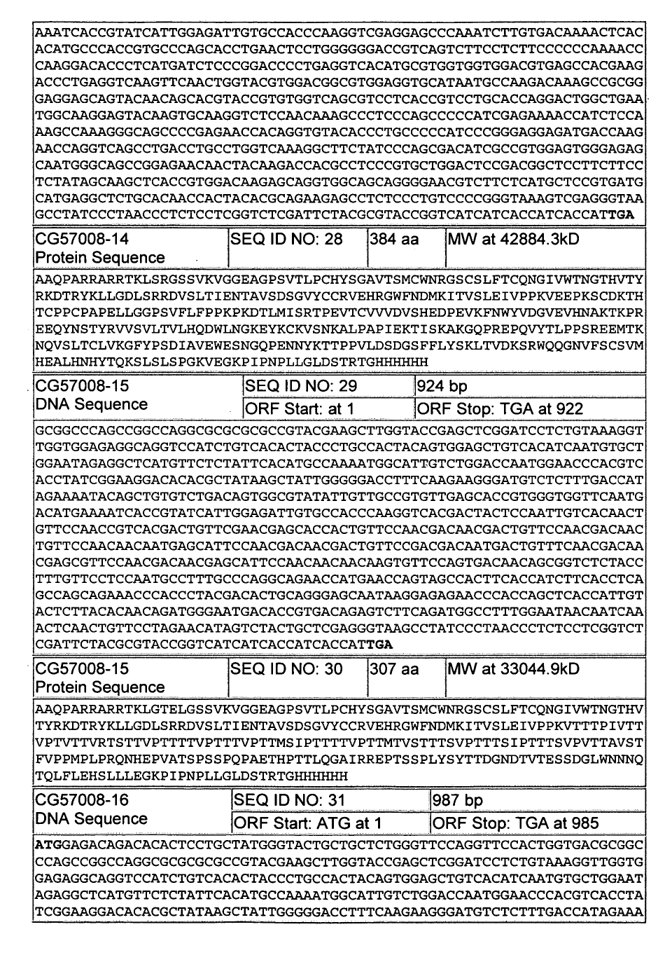

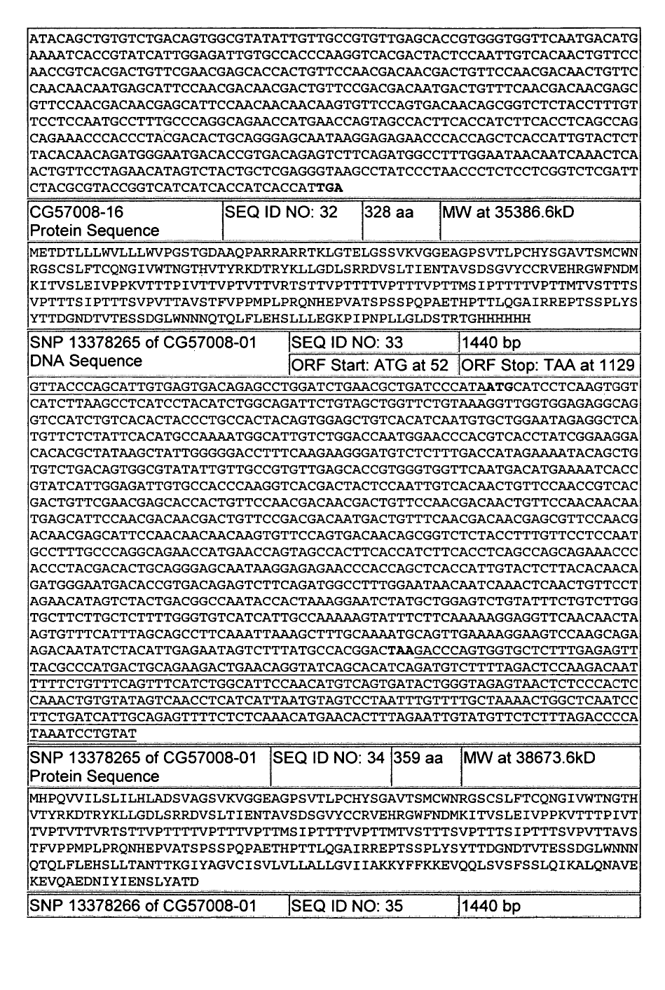

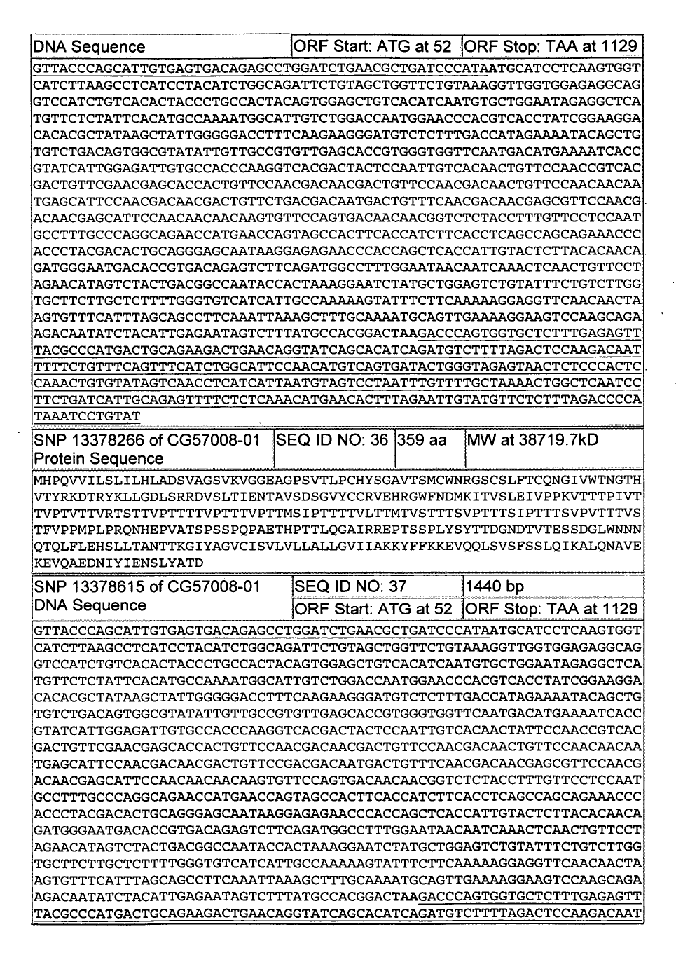

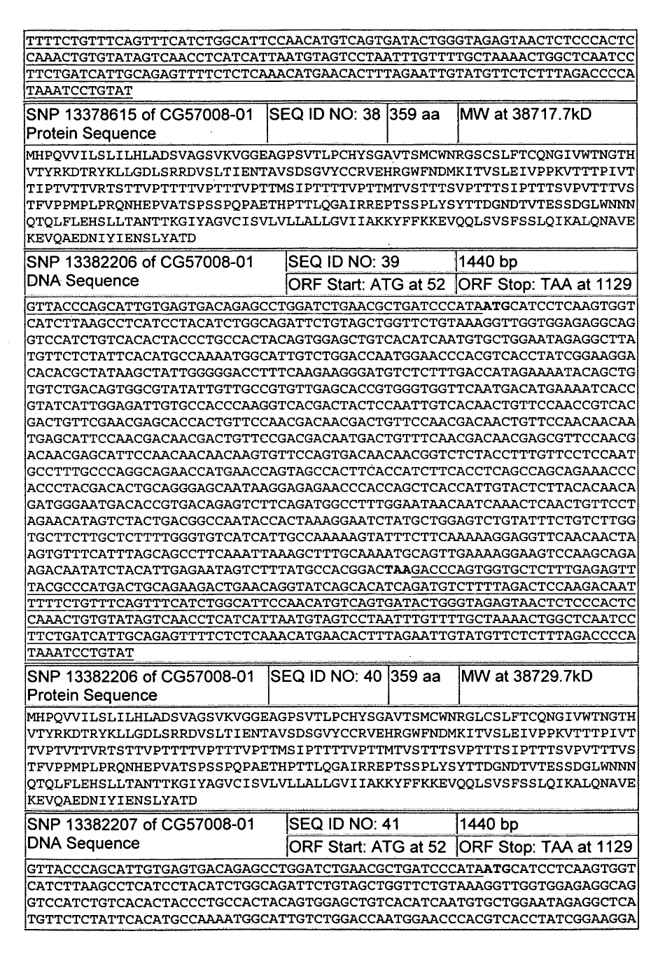

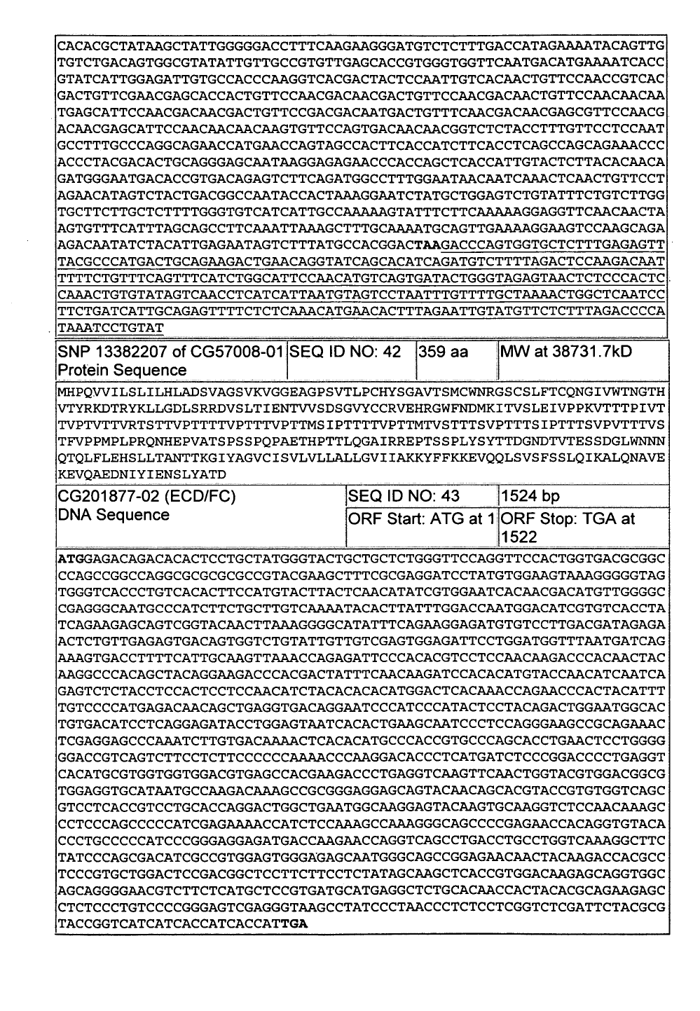

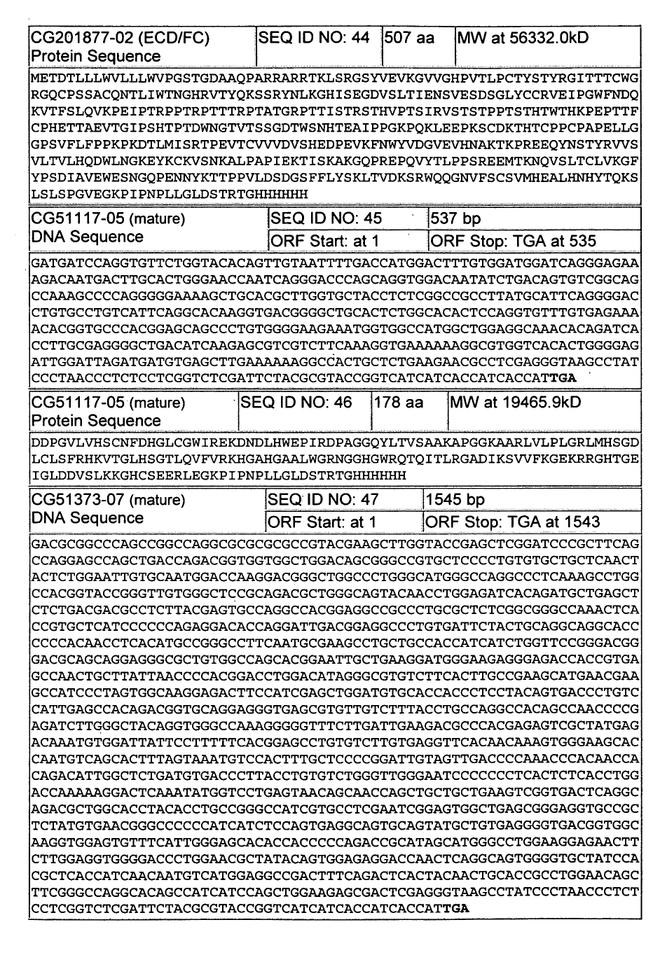

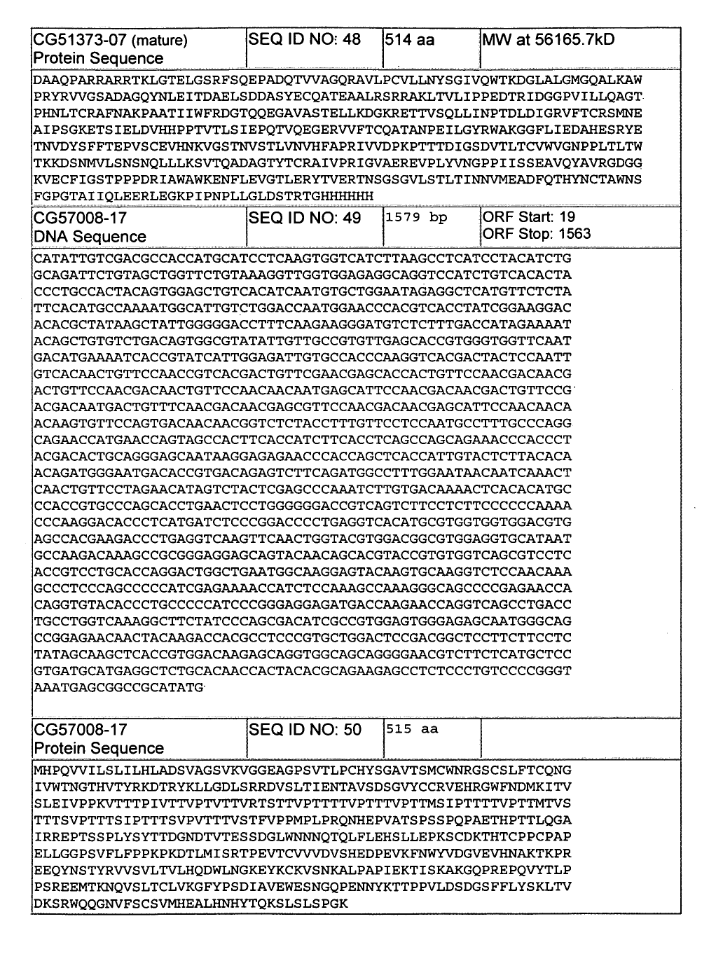

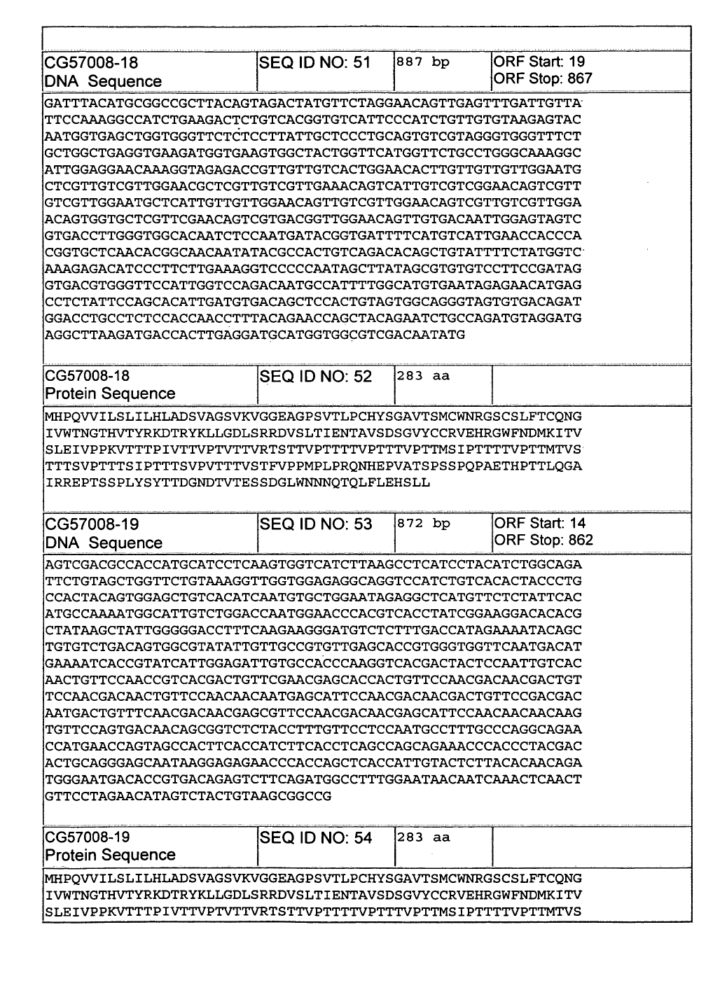

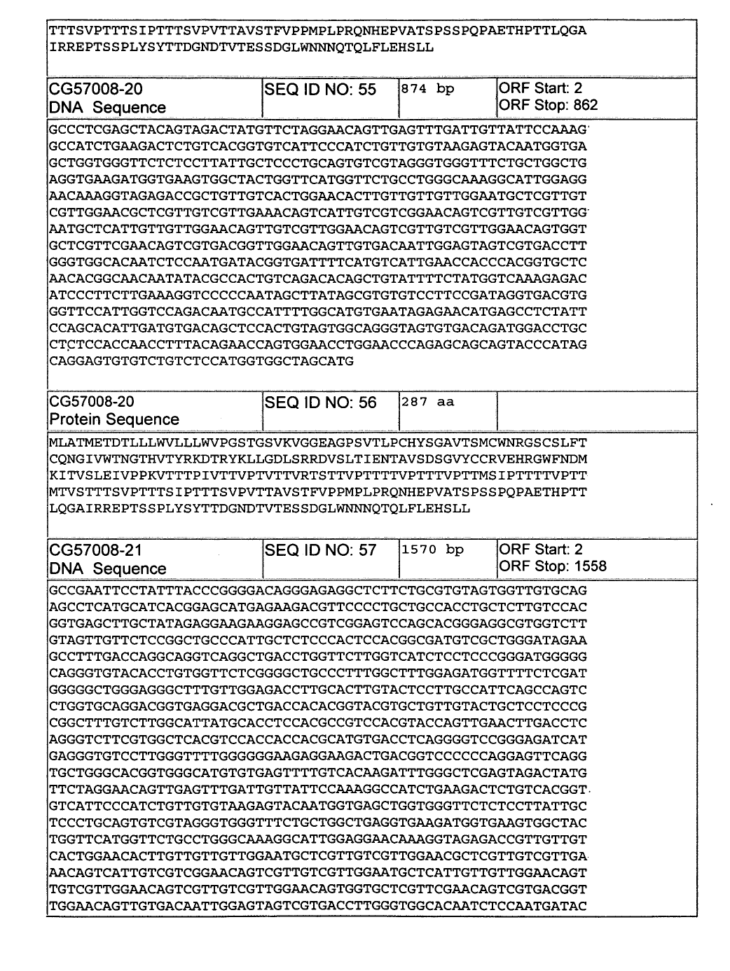

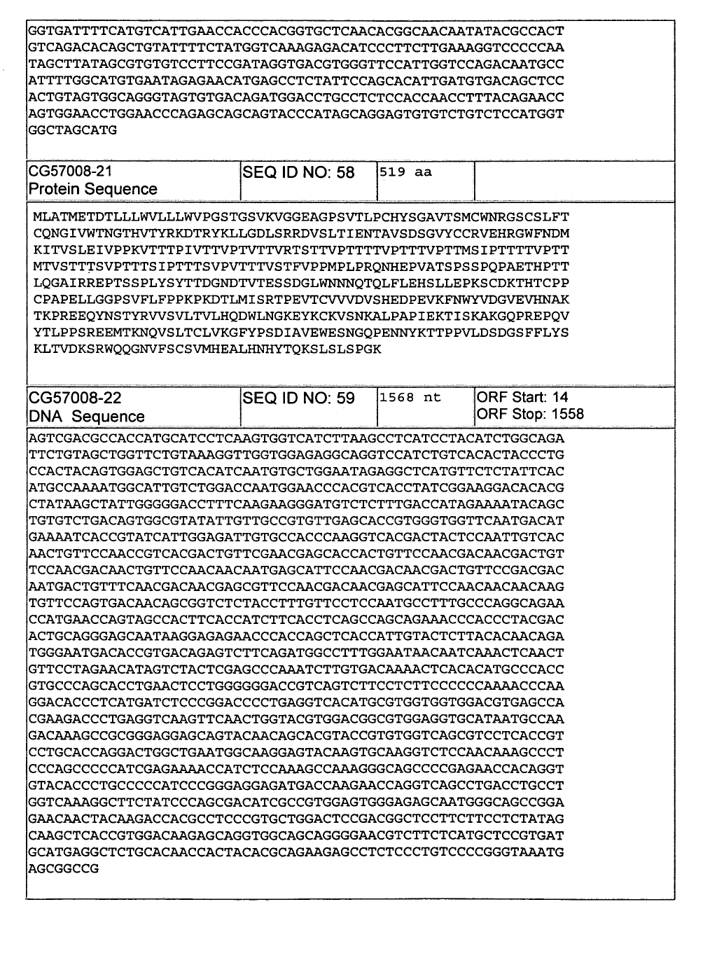

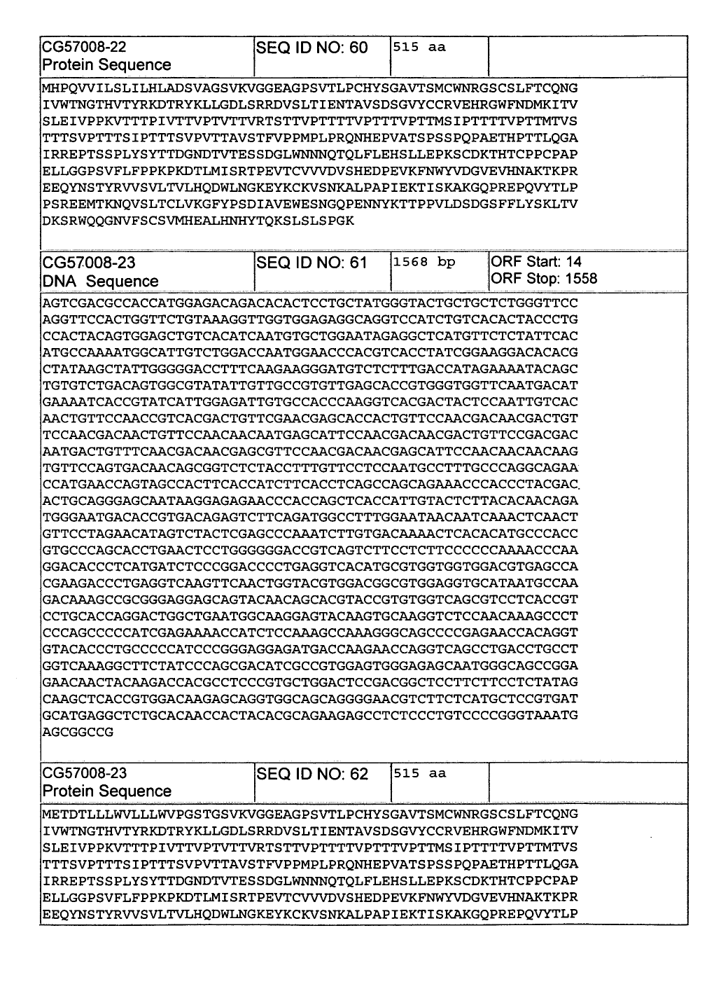

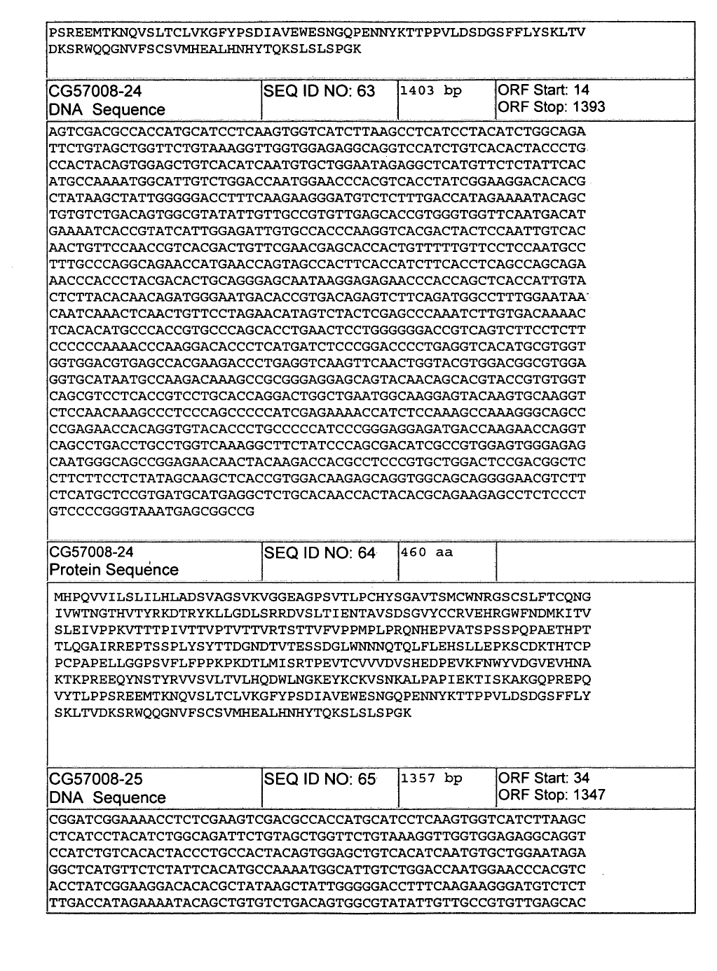

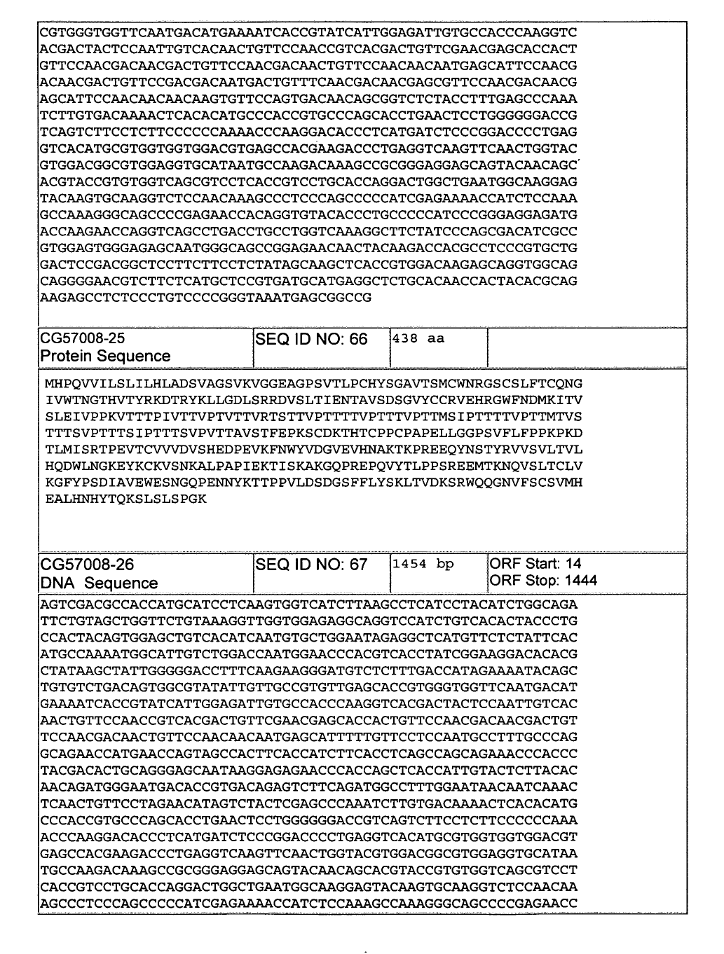

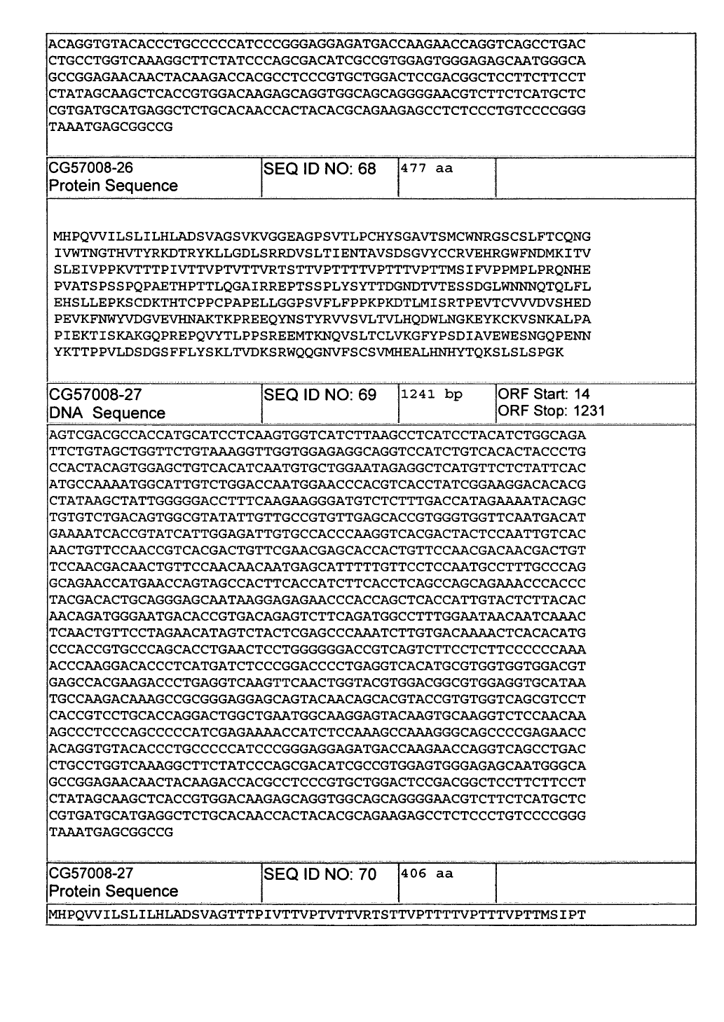

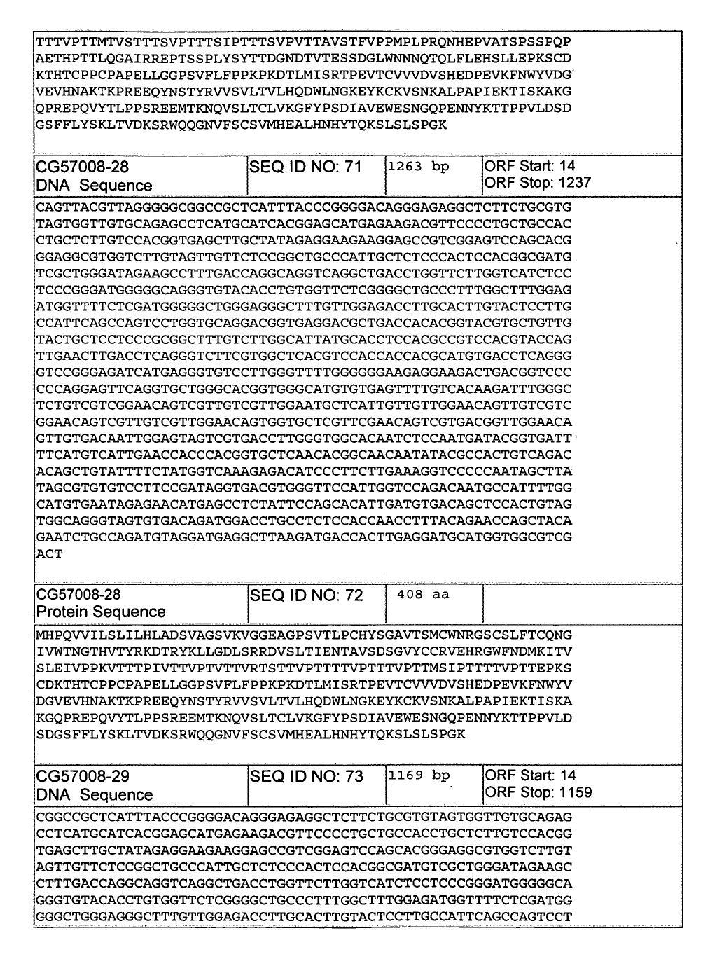

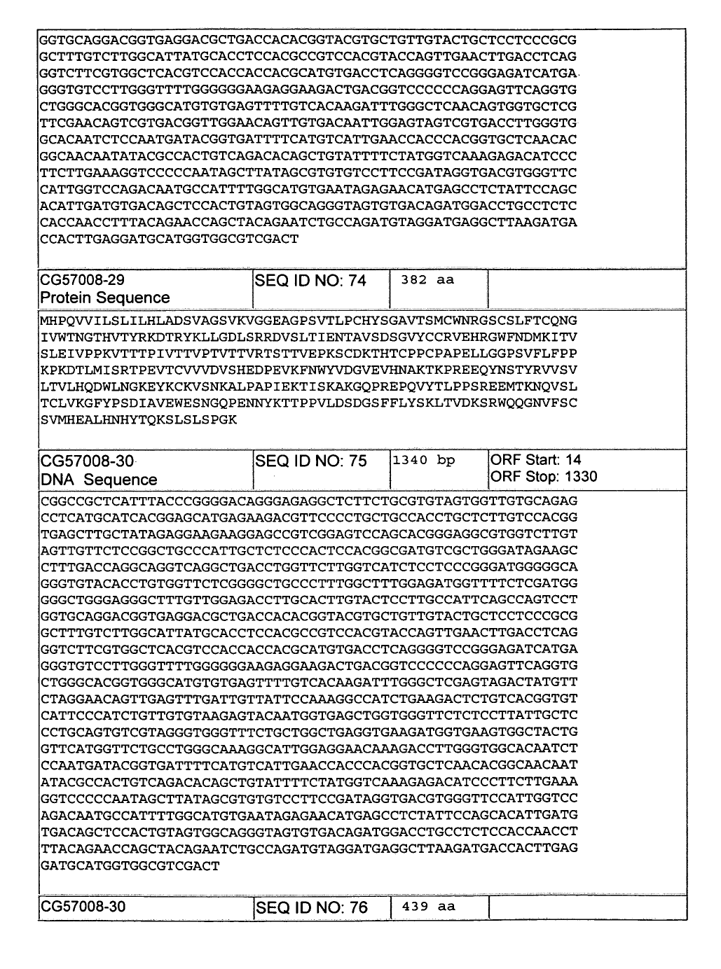

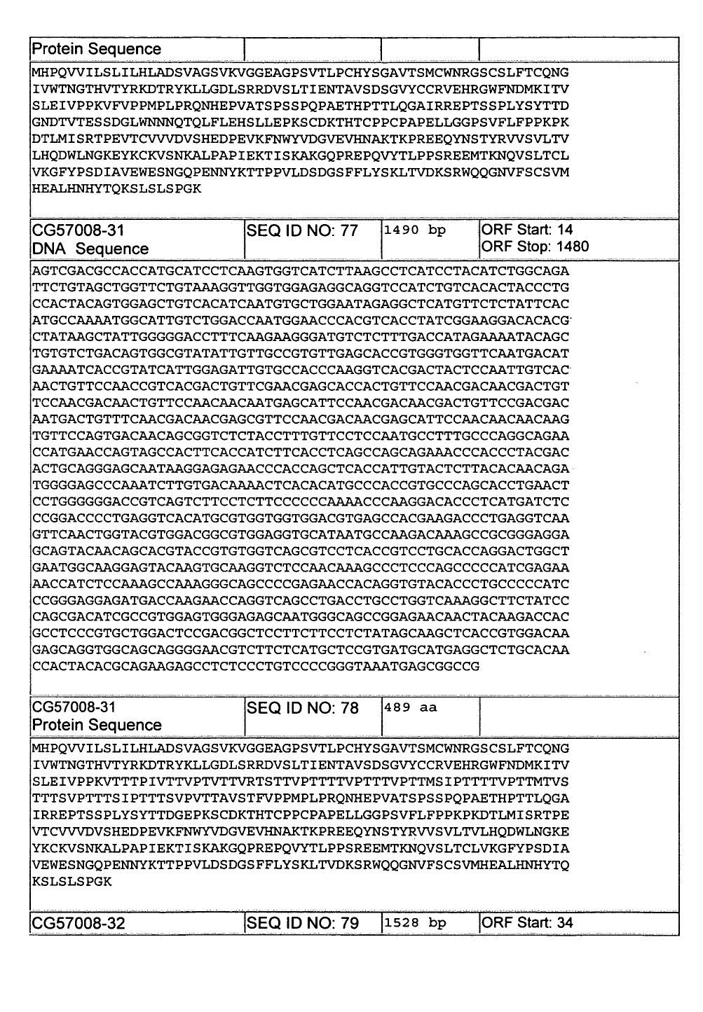

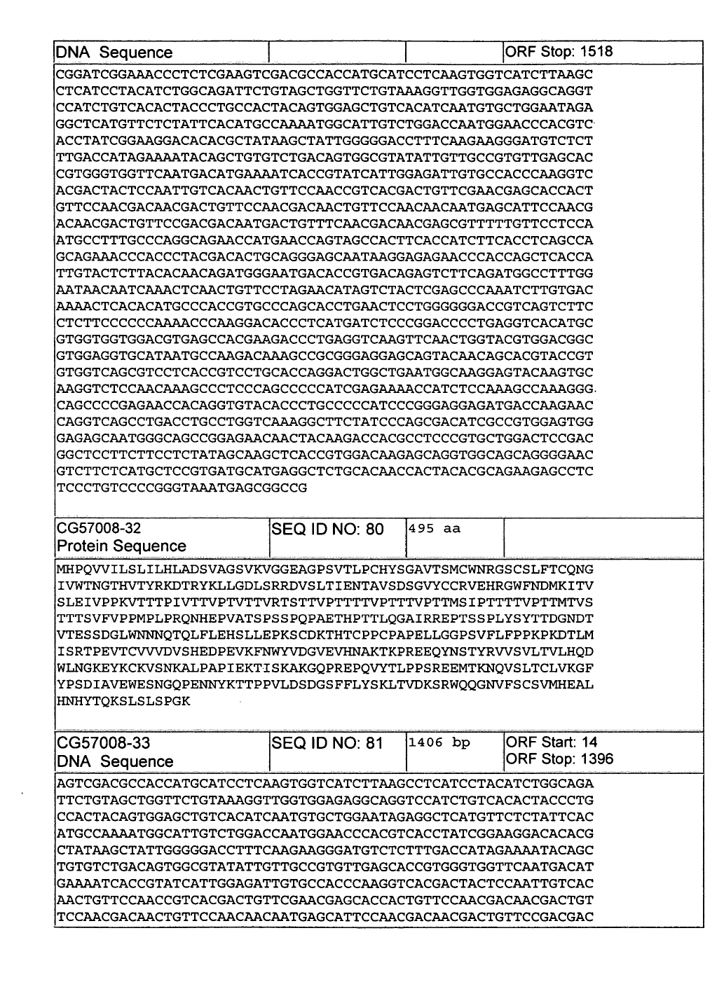

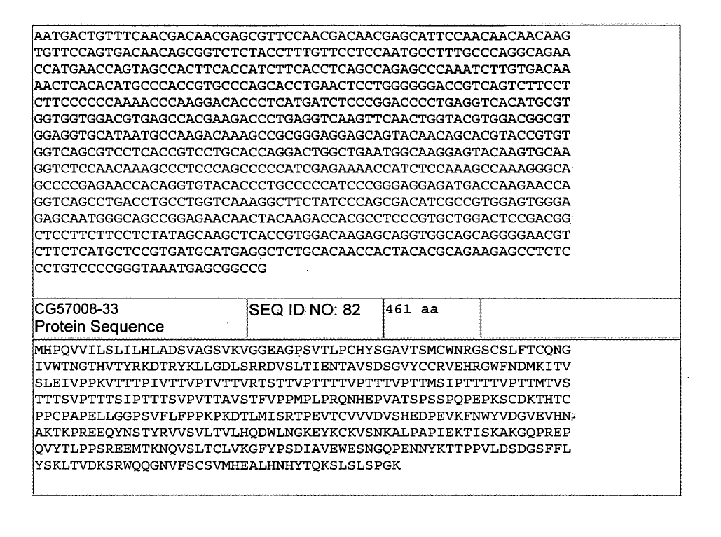

In some embodiments, CG57008 refers to CG57008-01 (SEQ ID NOs:1 and 2), CG57008-02 (SEQ ID NOs:3 and 4), CG57008-03 (SEQ ID NOs:5 and 2), CG57008-04 (SEQ ID NOs:6 and 7), CG57008-05 (SEQ ID NOs:8 and 9), CG57008-06 (SEQ ID NOs:10 and 11), CG57008-07 (SEQ ID NOs:12 and 13), CG57008-08 (SEQ ID NOs:14 and 15), CG57008-09 (SEQ ID NOs:16 and 17), CG57008-10 (SEQ ID NOs:18 and 19), CG57008-11 (SEQ ID NOs:20 and 21), CG57008-12 (SEQ ID NOs:22 and 23), CG57008-13 (SEQ ID NOs:24 and 25), CG57008-14 (SEQ ID NOs:26 and 27), CG57008-15 (SEQ ID NOs:28 and 29), CG57008-16 (SEQ ID NOs:30 and 31), CG57008-17 (SEQ ID NOs:32 and 33), CG57008-18 (SEQ ID NOs:34 and 35), CG57008-19 (SEQ ID NOs:36 and 37), CG57008-20 (SEQ ID NOs:38 and 39), CG57008-20 (SEQ ID NOs:40 and 41), CG57008-21 (SEQ ID NOs:42 and 43), CG57008-22 (SEQ ID NOs:44 and 45), CG57008-23 (SEQ ID NOs:46 and 47), CG57008-24 (SEQ ID NOs:48 and 49), CG57008-25 (SEQ ID NOs:50 and 51), CG57008-26 (SEQ ID NOs:52 and 53), CG57008-27 (SEQ ID NOs:54 and 55), CG57008-28 (SEQ ID NOs:56 and 57), CG57008-29 (SEQ ID NOs:58 and 59), CG57008-30 (SEQ ID NOs:60 and 61), CG57008-31 (SEQ ID NOs:62 and 63), CG57008-32 (SEQ ID NOs:64 and 65), CG57008-33 (SEQ ID NOs:66 and 67), or combinations thereof (See FIG. 10, a chart depicting sequence data for the various isoforms of CG57008).

### Methods of Preparing CG57008

Any techniques known in the art can be used in purifying a CG57008 protein, including but not limited to, separation by precipitation, separation by adsorption (e.g., column chromatography, membrane adsorbents, radial flow columns, batch adsorption, high-performance liquid chromatography, ion exchange chromatography, inorganic adsorbents, hydrophobic adsorbents, immobilized metal affinity chromatography, affinity chromatography), or separation in solution (e.g., gel filtration, electrophoresis, liquid phase partitioning, detergent partitioning, organic solvent extraction, and ultrafiltration). See e.g., Scopes, PROTEIN PURIFICATION, PRINCIPLES AND PRACTICE, 3rd ed., Springer (1994). During the purification, the biological activity of CG57008 may be monitored by one or more in vitro or in vivo assays. The purity of CG57008 can be assayed by any methods known in the art, such as but not limited to, gel electrophoresis. See Scopes, supra. In some embodiments, the CG57008 proteins employed in a composition of the invention can be in the range of 80 to 100 percent of the total mg protein, or at least 80%, at least 85%, at least 90%, at least 95%, or at least 98% of the total mg protein. In one embodiment, one or more CG57008 proteins employed in a composition of the invention is at least 99% of the total protein. In another embodiment, CG57008 is purified to apparent homogeneity, as assayed, e.g., by sodium dodecyl sulfate polyacrylamide gel electrophoresis.

Methods known in the art can be utilized to recombinantly produce CG57008 proteins. A nucleic acid sequence encoding a CG57008 protein can be inserted into an expression vector for propagation and expression in host cells.

An expression construct, as used herein, refers to a nucleic acid sequence encoding a CG57008 protein operably associated with one or more regulatory regions that enable expression of a CG57008 protein in an appropriate host cell. “Operably-associated” refers to an association in which the regulatory regions and the CG57008 sequence to be expressed are joined and positioned in such a way as to permit transcription, and ultimately, translation.

The regulatory regions that are necessary for transcription of CG57008 can be provided by the expression vector. A translation initiation codon (ATG) may also be provided if a CG57008 gene sequence lacking its cognate initiation codon is to be expressed. In a compatible host-construct system, cellular transcriptional factors, such as RNA polymerase, will bind to the regulatory regions on the expression construct to effect transcription of the modified CG57008 sequence in the host organism. The precise nature of the regulatory regions needed for gene expression may vary from host cell to host cell. Generally, a promoter is required which is capable of binding RNA polymerase and promoting the transcription of an operably-associated nucleic acid sequence. Such regulatory regions may include those 5′ non-coding sequences involved with initiation of transcription and translation, such as the TATA box, capping sequence, CAAT sequence, and the like. The non-coding region 3′ to the coding sequence may contain transcriptional termination regulatory sequences, such as terminators and polyadenylation sites.

In order to attach DNA sequences with regulatory functions, such as promoters, to a CG57008 gene sequence or to insert a CG57008 gene sequence into the cloning site of a vector, linkers or adapters providing the appropriate compatible restriction sites may be ligated to the ends of the cDNAs by techniques well known in the art (see e.g., Wu et al., 1987, Methods in Enzymol, 152:343-349). Cleavage with a restriction enzyme can be followed by modification to create blunt ends by digesting back or filling in single-stranded DNA termini before ligation. Alternatively, a desired restriction enzyme site can be introduced into a fragment of DNA by amplification of the DNA using PCR with primers containing the desired restriction enzyme site.

An expression construct comprising a CG57008 sequence operably associated with regulatory regions can be directly introduced into appropriate host cells for expression and production of a CG57008 protein without further cloning. See, e.g., U.S. Pat. No. 5,580,859. The expression constructs can also contain DNA sequences that facilitate integration of a CG57008 sequence into the genome of the host cell, e.g., via homologous recombination. In this instance, it is not necessary to employ an expression vector comprising a replication origin suitable for appropriate host cells in order to propagate and express CG57008 in the host cells.

A variety of expression vectors may be used, including but are not limited to, plasmids, cosmids, phage, phagemids or modified viruses. Such host-expression systems represent vehicles by which the coding sequences of a CG57008 gene may be produced and subsequently purified, but also represent cells which may, when transformed or transfected with the appropriate nucleotide coding sequences, express CG57008 in situ. These include, but are not limited to, microorganisms such as bacteria (e.g., E. coli and B. subtilis) transformed with recombinant bacteriophage DNA, plasmid DNA or cosmid DNA expression vectors containing CG57008 coding sequences; yeast (e.g., Saccharomyces, Pichia) transformed with recombinant yeast expression vectors containing CG57008 coding sequences; insect cell systems infected with recombinant virus expression vectors (e.g., baculovirus) containing CG57008 coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing CG57008 coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5 K promoter). Preferably, bacterial cells such as Escherichia coli and eukaryotic cells are used for the expression of a recombinant CG57008 molecule. For example, mammalian cells such as Chinese hamster ovary cells (CHO) can be used with a vector bearing promoter element from major intermediate early gene of cytomegalovirus for effective expression of a CG57008 sequence (Foecking et al., 1986, Gene 45:101; and Cockett et al., 1990, Bio/Technology 8:2).

In bacterial systems, a number of expression vectors may be advantageously selected depending upon the use intended for the CG57008 molecule being expressed. For example, when a large quantity of a CG57008 is to be produced, for the generation of pharmaceutical compositions of a CG57008 molecule, vectors that direct the expression of high levels of fusion protein products that are readily purified may be desirable. Such vectors include, but are not limited to, the E. coli expression vector pCR2.1 TOPO (Invitrogen); pIN vectors (Inouye & Inouye, 1985, Nucleic Acids Res. 13:3101-3109; Van Heeke & Schuster, 1989, J. Biol. Chem. 24:5503-5509) and the like. Series of vectors like pFLAG (Sigma), pMAL (NEB), and pET (Novagen) may also be used to express the foreign proteins as fusion proteins with FLAG peptide, malE-, or CBD-protein. These recombinant proteins may be directed into periplasmic space for correct folding and maturation. The fused part can be used for affinity purification of the expressed protein. Presence of cleavage sites for specific proteases like enterokinase allows one to cleave off the CG57008 protein. The pGEX vectors may also be used to express foreign proteins as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, many vectors to express foreign genes can be used, e.g., Autographa californica nuclear polyhedrosis virus (AcNPV) can be used as a vector to express foreign genes. The virus grows in cells like Spodoptera frugiperda cells. A CG57008 coding sequence may be cloned individually into non-essential regions (e.g., the polyhedrin gene) of the virus and placed under control of an AcNPV promoter (e.g., the polyhedrin promoter).

In mammalian host cells, a number of viral-based expression systems may be utilized. In cases where an adenovirus is used as an expression vector, a CG57008 coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region EI or E3) will result in a recombinant virus that is viable and capable of expressing CG57008 in infected hosts (see, e.g., Logan & Shenk, 1984, Proc. Natl. Acad. Sci. USA 8 1:355-359). Specific initiation signals may also be required for efficient translation of inserted CG57008 coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript and post-translational modification of the gene product, e.g., glycosylation and phosphorylation of the gene product, may be used. Such mammalian host cells include, but are not limited to, PC12, CHO, VERY, BHK, HeLa, COS, MDCK, 293, 3T3, W138, BT483, Hs578T, HTB2, BT2O and T47D, NS0 (a murine myeloma cell line that does not endogenously produce any immunoglobulin chains), CRL7O3O and HsS78Bst cells. Expression in a bacterial or yeast system can be used if post-translational modifications are found to be non-essential for a desired activity of CG57008.

For long-term, high-yield production of properly processed CG57008, stable expression in cells is preferred. Cell lines that stably express CG57008 may be engineered by using a vector that contains a selectable marker. By way of example but not limitation, following the introduction of the expression constructs, engineered cells may be allowed to grow for 1-2 days in an enriched media, and then are switched to a selective media. The selectable marker in the expression construct confers resistance to the selection and optimally allows cells to stably integrate the expression construct into their chromosomes and to grow in culture and to be expanded into cell lines. Such cells can be cultured for a long period of time while CG57008 is expressed continuously.

A number of selection systems may be used, including but not limited to, antibiotic resistance (markers like Neo, which confers resistance to geneticine, or G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIB TECH 11(5):I55-2 15); Zeo, for resistance to Zeocin; Bsd, for resistance to blasticidin, etc.); antimetabolite resistance (markers like Dhfr, which confers resistance to methotrexate, Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); and hygro, which confers resistance to hygromycin (Santerre et al., 1984, Gene 30:147). In addition, mutant cell lines including, but not limited to, tk-, hgprt- or aprt-cells, can be used in combination with vectors bearing the corresponding genes for thymidine kinase, hypoxanthine, guanine- or adenine phosphoribosyltransferase. Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1.

The recombinant cells may be cultured under standard conditions of temperature, incubation time, optical density and media composition. However, conditions for growth of recombinant cells may be different from those for expression of CG57008. Modified culture conditions and media may also be used to enhance production of CG57008. Any techniques known in the art may be applied to establish the optimal conditions for producing CG57008.

An alternative to producing CG57008 or a fragment thereof by recombinant techniques is peptide synthesis. For example, an entire CG57008, or a protein corresponding to a portion of CG57008, can be synthesized by use of a peptide synthesizer. Conventional peptide synthesis or other synthetic protocols well known in the art may be used.

Proteins having the amino acid sequence of CG57008 or a portion thereof may be synthesized by solid-phase peptide synthesis using procedures similar to those described by Merrifield, 1963, J. Am. Chem. Soc., 85:2149. During synthesis, N-α-protected amino acids having protected side chains are added stepwise to a growing polypeptide chain linked by its C-terminal and to an insoluble polymeric support, i.e., polystyrene beads. The proteins are synthesized by linking an amino group of an N-α-deprotected amino acid to an α-carboxyl group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc, which is acid labile, and Fmoc, which is base labile. Details of appropriate chemistries, resins, protecting groups, protected amino acids and reagents are well known in the art and so are not discussed in detail herein (See, Atherton et al., 1989, Solid Phase Peptide Synthesis:A Practical Approach, IRL Press, and Bodanszky, 1993, Peptide Chemistry, A Practical Textbook, 2nd Ed., Springer-Verlag).

Purification of the resulting CG57008 protein is accomplished using conventional procedures, such as preparative HPLC using gel permeation, partition and/or ion exchange chromatography. The choice of appropriate matrices and buffers are well known in the art and so are not described in detail herein.

Non-limiting examples of methods for preparing CG57008 can be found in Example 1, infra.

### Antibodies to CG57008

In various embodiments, monoclonal or polyclonal antibodies specific to CG57008, or a domain of CG57008, can be used in immunoassays to measure the amount of CG57008 or used in immunoaffinity purification of a CG57008 protein. A Hopp & Woods hydrophilic analysis (see Hopp & Woods, Proc. Natl. Acad. Sci. U.S.A. 78:3824-3828 (1981) can be used to identify hydrophilic regions of a protein, and to identify potential epitopes of a CG57008 protein.

The antibodies that immunospecifically bind to an CG57008 or an antigenic fragment thereof can be produced by any method known in the art for the synthesis of antibodies, in particular, by chemical synthesis or preferably, by recombinant expression techniques. (See, e.g., U.S. Ser. No. 10/805,177, which is incorporated herein in its entirety).

Polyclonal antibodies immunospecific for CG57008 or an antigenic fragment thereof can be produced by various procedures well-known in the art. For example, a CG57008 protein can be administered to various host animals including, but not limited to, rabbits, mice, and rats, to induce the production of sera containing polyclonal antibodies specific for the CG57008. Various adjuvants may be used to increase the immunological response, depending on the host species, including but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies:A Laboratory Manual, (Cold Spring Harbor Laboratory Press, 2nd ed. 1988); Hammerling et al., in:Monoclonal Antibodies and T Cell Hybridomas 563 681 (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with a non-murine antigen and once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

The present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with a non-murine antigen with myeloma cells and then screening the hybridomas resulting from the fusion for hybridoma clones that secrete an antibody able to bind to the antigen.

Antibody fragments which recognize specific particular epitopes may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with a scFv linker by PCR and cloned into a phagemid vector. The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; International application No. PCT/GB91/01 134; International publication Nos. WO 90/02809, WO 91/10737, WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108.

As described in the above references, after phage selection, the antibody coding regions from the phage can be isolated and used to generate whole antibodies or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in PCT publication No. WO 92/22324; Mullinax et al., 1992, BioTechniques 12(6):864-869; Sawai et al., 1995, AJRI34:26-34; and Better et al., 1988, Science 240:1041-1043.

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lambda constant regions. Preferably, the vectors for expressing the VH or VL domains comprise an EF-1α promoter, a secretion signal, a cloning site for the variable domain, constant domains, and a selection marker such as neomycin. The VH and VL domains may also cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use humanized antibodies or chimeric antibodies. Human antibodies can be made by a variety of methods known in the art including phage display methods described above using antibody libraries derived from human immunoglobulin sequences. See also U.S. Pat. Nos. 4,444,887 and 4,716,111; and International publication Nos. WO 98/46645, WO 98/50433, WO 98/24893, WO98/16654, WO 96/34096, WO 96/33735, and WO 91/10741.

A chimeric antibody is a molecule in which different portions of the antibody are derived from different immunoglobulin molecules. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,8 16397, and 6,311,415.

A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non human immunoglobulin. A humanized antibody comprises substantially all of at least one, and typically two, variable domains (Fab, Fab′, F(ab′)2, Fabc, Fv) in which all or substantially all of the CDR regions correspond to those of a non human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG1, IgG2, IgG3 and IgG4. Usually the constant domain is a complement fixing constant domain where it is desired that the humanized antibody exhibit cytotoxic activity, and the class is typically IgG1. Where such cytotoxic activity is not desirable, the constant domain may be of the IgG2 class. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental framework region (FR) and CDR sequences, more often 90%, and most preferably greater than 95%. Humanized antibody can be produced using variety of techniques known in the art, including but not limited to, CDR grafting (European Patent No. EP 239,400; International Publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489 498; Studnicka et al., 1994, Protein Engineering 7(6):805 814; and Roguska et al., 1994, PNAS 91:969 973), chain shuffling (U.S. Pat. No. 5,565,332), and techniques disclosed in, e.g., U.S. Pat. No. 6,407,213, U.S. Pat. No. 5,766,886, WO 9317105, Tan et al., J. Immunol. 169:1119 25 (2002), Caldas et al., Protein Eng. 13(5):353 60 (2000), Morea et al., Methods 20(3):267 79 (2000), Baca et al., J. Biol. Chem. 272(16):10678 84 (1997), Roguska et al., Protein Eng. 9(10):895 904 (1996), Couto et al., Cancer Res. 55 (23 Supp):5973s 5977s (1995), Couto et al., Cancer Res. 55(8):1717 22 (1995), Sandhu J S, Gene 150(2):409 10 (1994), and Pedersen et al., J. Mol. Biol. 235(3):959 73 (1994). Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323.)

### Characterization and Demonstration of CG57008 Activities and Monitoring Effects During Treatment

Any methods known in the art can be used to determine the identity of a purified CG57008 protein of the instant invention. Such methods include, but are not limited to, Western Blot, sequencing (e.g., Edman sequencing), liquid chromatography (e.g., HPLC, RP-HPLC with both UV and electrospray mass spectrometric detection), mass spectrometry, total amino acid analysis, peptide mapping, and SDS-PAGE. The secondary, tertiary and/or quaternary structure of a CG57008 protein can analyzed by any methods known in the art, e.g., far UV circular dichroism spectrum can be used to analyze the secondary structure, near UV circular dichroism spectroscopy and second derivative UV absorbance spectroscopy can be used to analyze the tertiary structure, and light scattering SEC-HPLC can be used to analyze quaternary structure.

The purity of a CG57008 protein of the instant invention can be analyzed by any methods known in the art, such as but not limited to, sodium dodecyl sulphate polyacrylamide gel electrophoresis (“SDS-PAGE”), reversed phase high-performance liquid chromatography (“RP-HPLC”), size exclusion high-performance liquid chromatography (“SEC-HPLC”), and Western Blot (e.g., host cell protein Western Blot). In a preferred embodiment, a CG57008 protein in a composition used in accordance to the instant invention is 80%-100% pure by densitometry, or at least 97%, at least 98%, or at least 99% pure by densitometry. In another preferred embodiment, a CG57008 protein in a composition used in accordance to the instant invention is more than 97%, more than 98%, or more than 99% pure by densitometry.

The biological activities and/or potency of CG57008 of the present invention can be determined by any methods known in the art. For example, compositions for use in therapy in accordance to the methods of the present invention can be tested in suitable cell lines for one or more activities that TIM-1 possesses.

Structure prediction, analysis of crystallographic data, sequence alignment, as well as homology modeling, can also be accomplished using computer software programs available in the art, such as BLAST, CHARMm release 21.2 for the Convex, and QUANTA v.3.3, (Molecular Simulations, Inc., York, United Kingdom). Other methods of structural analysis can also be employed. These include, but are not limited to, X-ray crystallography (Engstom, A., 1974, Biochem. Exp. Biol. 11:7-13) and computer modeling (Fletterick, R. and Zoller, M. (eds.), 1986, Computer Graphics and Molecular Modeling, in Current Communications in Molecular Biology, Cold Spring Harbor Laboratory, Cold Spring Harbor, N.Y.).

The half-life of a protein is a measurement of protein stability and indicates the time necessary for a one half reduction in activity of the protein. The half-life of a CG57008 protein can be determined by any method measuring activity of CG57008 in samples from a subject over a period of time. The normalization to concentration of CG57008 in the sample can be done by, e.g., immunoassays using anti-CG57008 antibodies to measure the levels of the CG57008 molecules in samples taken over a period of time after administration of the CG57008, or detection of radiolabelled CG57008 molecules in samples taken from a subject after administration of the radiolabeled CG57008 molecules. In specific embodiments, techniques known in the art can be used to prolong the half life of an CG57008 in vivo. For example, albumin or inert polymer molecules such as high molecular weight polyethyleneglycol (PEG) can be used. See, e.g., International Publication Nos. WO 93/15199, WO 93/15200, and WO 01/77137; and U.S. Pat. No. 6,528,485.

Compositions comprising one more CG57008 for use in a therapy can also be tested in suitable animal model systems prior to testing in humans. To establish an estimate of drug activity in relevant model experiments, an index can be developed that combines observational examination of the animals as well as their survival status. The effectiveness of CG57008 in preventing and/or treating a disease can be monitored by any methods known to one skilled in the art.

Therapeutic Uses

The methods of the present invention are based, in part, upon the discovery that CG57008 polypeptides inhibit T-cell activation/proliferation and/or cytokine release from immune cells.

In one embodiment, the invention provides a method for inhibiting the activation and/or proliferation of immune cells. The method includes contacting immune cells with a composition comprising a CG57008 polypeptide. In a preferred embodiment, the immune cells that are inhibited by CG57008 are T-cells, including helper T-cells (e.g., CD4+ or CD8+ T-cells), Th1 cells, or Th2 cells.

In another embodiment, the invention provides a method of inhibiting cytokine release from immune cells. The method includes contacting immune cells (e.g., T-helper cells (including CD4+ T-cells and CD8+ T-cells) or T_{H}2 cells) with a composition comprising a CG57008 polypeptide. The cytokine may be IFN-gamma, a member of the interleukin family (e.g., IL-2, IL-5, or IL-10), Tumor necrosis factor (TFN), or a combination thereof.

In another embodiment, the invention provides a method of treating a pathological state in a subject, wherein the pathological state results from an improper immune response. The method includes administering to the subject a protein of the invention in an amount that is sufficient to alleviate the pathological state, wherein the CG57008 protein is a protein having an amino acid sequence at least 85%, 90%, 95%, 96%, 97%, 98%, or even 99% identical to a protein comprising an amino acid sequence of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, and 82, or a biologically active fragment thereof. In another related method, an antibody of the invention is administered to the subject.

T-Cell Mediated Diseases

In a specific embodiment, the present invention provides a method for treating T-cell mediated diseases, in particular, T-cell mediated diseases that result from T-cell activation and/or proliferation. The method includes administering to a subject a CG57008 composition in an amount and for a duration that is effective to inhibit T-cell activation and/or proliferation.

Examples of T-cell mediated diseases that may be treated and/or prevented by the administration of a CG57008 composition include but are not limited to, atopic conditions (IgE-mediated allergic conditions), such as asthma, allergy, including allergic rhinitis, dermatitis, including psoriasis, pathogen susceptibilities, chronic inflammatory disease, organ-specific autoimmunity including multiple sclerosis, Hashimoto's thyroiditis and Grave's disease, graft rejection, and graft-versus-host disease. Other immune disorders involving T cell activation include, but are not limited to, chronic inflammatory diseases and disorders, such as Crohn's disease, systemic lupus erythematosus, myasthenia gravis, thyroiditis, reactive arthritis, including Lyme disease, rheumatoid arthritis, insulin-dependent diabetes, contact dermatitis, gastrointestinal allergies, including food allergies, eosinophilia, conjunctivitis, glomerular nephritis, certain pathogen susceptibilities such as helminthic (leishmaniasis), gram positive superantigen-induced shock, and certain viral infections, including HIV, and bacterial infections, including tuberculosis and lepromatous leprosy.

In a specific embodiment, the invention provides a method of treating Th2-mediated diseases, such as asthma or lupus. The method includes administering to the subject a CG57008 protein or antibody of the invention in an amount and for a duration that are effective to treat a Th2-mediated disease by blocking and/or inhibiting the production of Th-2 from immune cells.

Cytokine-Mediated Diseases

It is shown herein that CG57008 proteins inhibit the production of cytokines such as IFN-gamma, and various members of the IL family (e.g., IL-2, IL-5, and IL-10) from immune cells. Accordingly, the compounds of the invention are useful in the prevention and/or treatment of cytokine-mediated diseases involving the overproduction of cytokines from immune cells.

Cytokine-mediated diseases and disorders that are associated with the overproduction of cytokines, and thus are within the scope of the present invention include, but are not limited to, inflammatory and allergic diseases, for example inflammation of the joints (especially rheumatoid arthritis, osteoarthritis and gout), inflammation of the gastrointestinal tract (especially inflammatory bowel disease, ulcerative colitis, Crohn's disease and gastritis), skin disease (especially psoriasis, eczema and dermatitis) and respiratory disease (especially asthma, bronchitis, allergic rhinitis and adult respiratory distress syndrome), and in the production and development of various cardiovascular and cerebrovascular disorders such as congestive heart failure, myocardial infarction, the formation of atherosclerotic plaques, hypertension, platelet aggregation, angina, stroke, reperfusion injury, vascular injury including restenosis and peripheral vascular disease, and, for example, various disorders of bone metabolism such as osteoporosis (including senile and postmenopausal osteoporosis), Paget's disease, bone metastases, hypercalcaemia, hyperparathyroidism, osteosclerosis, osteoperosis and periodontitis, and the abnormal changes in bone metabolism which may accompany rheumatoid arthritis and osteoarthritis. Excessive cytokine production has also been implicated in mediating certain complications of bacterial, fungal and/or viral infections such as endotoxic shock, septic shock and toxic shock syndrome and in mediating certain complications of CNS surgery or injury such as neurotrauma and ischaemic stroke. Excessive cytokine production has also been implicated in mediating or exacerbating the development of diseases involving cartilage or muscle resorption, pulmonary fibrosis, cirrhosis, renal fibrosis, the cachexia found in certain chronic diseases such as malignant disease and acquired immune deficiency syndrome (AIDS), tumour invasiveness and tumour metastasis and multiple sclerosis.

Accordingly, use of the CG57008 proteins to inhibit the production of and/or effects of these cytokines will be of benefit in the prevention and/or treatment of cytokine-mediated diseases and medical conditions.

In one embodiment, the present invention provides a method of preventing and/or treating cytokine-mediated inflammatory and/or autoimmune diseases. The method includes administering to a subject a CG57008 protein of the invention in an amount and for a duration that is effective to inhibit cytokine release from immune cells (e.g., T-helper cells (including CD4+ T-cells and CD8+ T-cells) or T_{H}2 cells). In some embodiments, the cytokines to be inhibited by CG57008 include, but are not limited to IFN-gamma and interleukins (e.g., IL-2, IL-5, and IL-10).

For example, in a further embodiment, the present invention provides a method of preventing and/or treating an IFN-gamma-mediated disease (e.g., inflammatory diseases, such as inflammatory bowel disease), by inhibiting the release of IFN-gamma from immune cells. The method includes administering to a subject in need thereof, an effective amount of a CG57008 protein.

In another embodiment, the present invention provides a method of preventing and/or treating an interleukin-mediated disease by inhibiting the release of interleukins from immune cells. The method includes administering to a subject in need thereof, an effective amount of a CG57008 protein.

In another embodiment, the present invention provides a method of preventing and/or treating an IL-2 mediated disease (e.g., autoimmune diseases (such as rheumatoid arthritis, multiple sclerosis, uveitis, psoriasis, arthritis, Type I insulin-dependent diabetes, Hashimoto's thyroiditis, Grave's thyroiditis, autoimmune myocarditis), allergic disorders such as hay fever, extrinsic asthma, or insect bite and sting allergies, food and drug allergies, as well as for the treatment or prevention of graft rejection) by inhibiting the release of IL-2 from immune cells. The method includes administering to a subject in need thereof, an effective amount of a CG57008 protein.

In another embodiment, the present invention provides a method of preventing and/or treating an IL-5 mediated disease (e.g., asthma and eczema) by inhibiting the release of IL-5 from immune cells. The method includes administering to a subject in need thereof, an effective amount of a CG57008 protein.

In another embodiment, the present invention provides a method of preventing and/or treating an IL-10 mediated disease (e.g., microbially induced shock or control B cell differentiation or development) by inhibiting the release of IL-10 from immune cells. The method includes administering to a subject in need thereof, an effective amount of a CG57008 protein.

Inflammatory Diseases/Disorders

In another aspect, the invention provides methods of preventing and/or treating inflammatory diseases/disorders. The method includes administering to a subject, in need thereof, a CG57008 protein of the invention in an amount and for a duration that are effective to prevent and/or treat the inflammatory disease. In a specific embodiment, the inflammatory disease to be treated and/or prevented by the administration of a CG57008 protein is arthritis (such as, antigen-induced arthritis or rheumatoid arthritis). In another embodiment, the inflammatory disease to be prevented and/or treated is contact hypersensitivity or contact dermatitis.

Autoimmune Disorders

In another aspect, the invention provides a method of diagnosing, treating, preventing or delaying an autoimmune disorder. The method includes administering to a subject, in need thereof, a CG57008 protein of the invention in an amount and for a duration that are effective to prevent and/or treat the autoimmune disease.

In accordance with the methods of the instant invention, the CG57008 composition comprises a protein selected from the group consisting of:SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, or combinations thereof. In some embodiments, the CG57008 composition is CG57008-01 (SEQ ID NOs:1 and 2), CG57008-02 (SEQ ID NOs:3 and 4), CG57008-03 (SEQ ID NOs:5 and 2), CG57008-04 (SEQ ID NOs:6 and 7), CG57008-05 (SEQ ID NOs:8 and 9), CG57008-06 (SEQ ID NOs:10 and 11), CG57008-07 (SEQ ID NOs:12 and 13), CG57008-08 (SEQ ID NOs:14 and 15), CG57008-09 (SEQ ID NOs:16 and 17), CG57008-10 (SEQ ID NOs:18 and 19), CG57008-11 (SEQ ID NOs:20 and 21), CG57008-12 (SEQ ID NOs:22 and 23), CG57008-13 (SEQ ID NOs:24 and 25), CG57008-14 (SEQ ID NOs:26 and 27), CG57008-15 (SEQ ID NOs:28 and 29), CG57008-16 (SEQ ID NOs:30 and 31), CG57008-17 (SEQ ID NOs:32 and 33), CG57008-18 (SEQ ID NOs:34 and 35), CG57008-19 (SEQ ID NOs:36 and 37), CG57008-20 (SEQ ID NOs:38 and 39), CG57008-20 (SEQ ID NOs:40 and 41), CG57008-21 (SEQ ID NOs:42 and 43), CG57008-22 (SEQ ID NOs:44 and 45), CG57008-23 (SEQ ID NOs:46 and 47), CG57008-24 (SEQ ID NOs:48 and 49), CG57008-25 (SEQ ID NOs:50 and 51), CG57008-26 (SEQ ID NOs:52 and 53), CG57008-27 (SEQ ID NOs:54 and 55), CG57008-28 (SEQ ID NOs:56 and 57), CG57008-29 (SEQ ID NOs:58 and 59), CG57008-30 (SEQ ID NOs:60 and 61), CG57008-31 (SEQ ID NOs:62 and 63), CG57008-32 (SEQ ID NOs:64 and 65), CG57008-33 (SEQ ID NOs:66 and 67), or combinations thereof.

The amount of the composition of the invention which will be effective in the treatment of a particular disorder or condition will depend on the nature of the disorder or condition, and can be determined by standard clinical techniques. The precise dose to be employed in the formulation will also depend on the route of administration, and the seriousness of the disease or disorder, and should be decided according to the judgment of the practitioner and each patient's circumstances.

In some embodiments, a composition comprising one or more isolated CG57008 proteins can also be used in combination with other therapies to prevent and/or treat the diseases/disorders referenced above. In one embodiment, a composition comprising one or more isolated CG57008 proteins is administered in combination with one or more other agents that have prophylactic and/or therapeutic effect(s) on a disease and/or have amelioration effect(s) on one or more symptoms associated with the disease to a subject to prevent and/or treat the disease. Any other agents or therapies that are known in the art that can be used to prevent and/or treat a disease, such as a T-cell mediated disease, a cytokine mediated disease, an inflammatory disease (such as arthritis), or an autoimmune disease, can be used in combination with a composition comprising one or more CG57008 proteins in accordance to the methods of the present invention.

Toxicity and efficacy of the prophylactic and/or therapeutic protocols of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

### Administration, Formulations and Kits

The compositions used in accordance to methods of the invention can be administered to a subject at a prophylactically or therapeutically effective amount to prevent and/or treat T-cell mediated diseases (including inflammatory diseases, such as arthritis, contact hypersensitivity, and contact dermatitis). Various delivery systems are known and can be used to administer a composition used in accordance to the methods of the invention. Such delivery systems include, but are not limited to, encapsulation in liposomes, microparticles, microcapsules, expression by recombinant cells, receptor-mediated endocytosis, construction of the nucleic acids of the invention as part of a retroviral or other vectors, etc. Methods of introduction include, but are not limited to, intradermal, intramuscular, intraperitoneal, intrathecal, intracerebroventricular, epidural, intravenous, subcutaneous, intranasal, intratumoral, transdermal, transmucosal, rectal, and oral routes. The compositions used in accordance to the methods of the invention may be administered by any convenient route, for example, by infusion or bolus injection, by absorption through epithelial or mucocutaneous linings (e.g., eye mucosa, oral mucosa, vaginal mucosa, rectal and intestinal mucosa, etc.), and may be administered together with other biologically active agents. Administration can be systemic or local. In a specific embodiment, the present invention comprises using single or double chambered syringes, preferably equipped with a needle-safety device and a sharper needle, that are pre-filled with a composition comprising one or more CG57008 proteins. In one embodiment, dual chambered syringes (e.g., Vetter Lyo-Ject dual-chambered syringe by Vetter Pharmar-Fertigung) are used. Such systems are desirable for lyophilized formulations, and are especially useful in an emergency setting.

In some embodiments, it may be desirable to administer the pharmaceutical compositions of the invention locally to the area in need of treatment. This may be achieved by, for example, local infusion during surgery, or topical application, e.g., in conjunction with a wound dressing after surgery, by injection, by means of a catheter, by means of a suppository, or by means of an implant (said implant being of a porous, non-porous, or gelatinous material, including membranes, such as sialastic membranes, or fibers). In one embodiment, administration can be by direct injection at the site (or former site) of rapidly proliferating tissues that are most sensitive to an insult, such as radiation, chemotherapy, or chemical/biological warfare agent.

In some embodiments, where the composition of the invention is a nucleic acid encoding a prophylactic or therapeutic agent, the nucleic acid can be administered in vivo to promote expression of their encoded proteins (e.g., CG57008 proteins), by constructing the nucleic acid as part of an appropriate nucleic acid expression vector and administering it so that it becomes intracellular, e.g., by use of a retroviral vector, or by direct injection, or by use of microparticle bombardment (e.g., a gene gun), or coating with lipids or cell-surface receptors or transfecting agents, or by administering it in linkage to a homeobox-like peptide which is known to enter the nucleus, etc. Alternatively, a nucleic acid of the invention can be introduced intracellularly and incorporated within host cell DNA for expression, by homologous recombination.

The instant invention encompasses bulk drug compositions useful in the manufacture of pharmaceutical compositions that can be used in the preparation of unit dosage forms. In a preferred embodiment, a composition of the invention is a pharmaceutical composition. Such compositions comprise a prophylactically or therapeutically effective amount of CG57008, and a pharmaceutically acceptable carrier. Preferably, the pharmaceutical compositions are formulated to be suitable for the route of administration to a subject.

In one embodiment, the term “pharmaceutically acceptable” means approved by a regulatory agency of the Federal or a state government or listed in the U.S. Pharmacopeia or other generally regarded as safe for use in humans (GRAS). The term “carrier” refers to a diluent, adjuvant, bulking agent (e.g., arginine in various salt forms, sulfobutyl ether Beta-cyclodextrin sodium, or sucrose), excipient, or vehicle with which CG57008 is administered. Such pharmaceutical carriers can be sterile liquids, such as water and oils (e.g., oils of petroleum, animal, vegetable or synthetic origins, such as peanut oil, soybean oil, mineral oil, sesame oil and the like), or solid carriers, such as one or more substances which may also act as diluents, flavoring agents, solubilizers, lubricants, suspending agents, or encapsulating material. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include, but are not limited to, starch or its synthetically modified derivatives such as hydroxyethyl starch, stearate salts, glycerol, glucose, lactose, sucrose, trehalose, gelatin, sulfobutyl ether Beta-cyclodextrin sodium, sodium chloride, glycerol, propylene, glycol, water, ethanol, or a combination thereof. The composition, if desired, can also contain minor amounts of wetting or emulsifying agents, or pH buffering agents.

The compositions comprising CG57008 may be formulated into any of many possible dosage forms such as, but not limited to, liquid, suspension, microemulsion, microcapsules, tablets, capsules, gel capsules, soft gels, pills, powders, enemas, sustained-release formulations and the like. The compositions comprising CG57008 may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. The composition can also be formulated as a suppository, with traditional binders and carriers such as triglycerides. Oral formulation can include standard carriers, such as pharmaceutical grades of mannitol, lactose, starch or its synthetically modified derivatives such as hydroxyethyl starch, stearate salts, sodium saccharine, cellulose, magnesium carbonate, etc.

A pharmaceutical composition comprising CG57008 is formulated to be compatible with its intended route of administration. In a specific embodiment, the composition is formulated in accordance with routine procedures as a pharmaceutical composition adapted for intravenous, subcutaneous, intramuscular, oral, intranasal, intratumoral or topical administration to human beings. Typically, compositions for intravenous administration are solutions in sterile isotonic or hypertonic aqueous buffer. Where necessary, the composition may also include a solubilizing agent and a local anesthetic such as benzyl alcohol or lidocaine to ease pain at the site of the injection.

If a composition comprising CG57008 is to be administered topically, the composition can be formulated in the form of transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Preferred topical formulations include those in which the compositions of the invention are in admixture with a topical delivery agent, such as but not limited to, lipids, liposomes, fatty acids, fatty acid esters, steroids, chelating agents and surfactants. The compositions comprising CG57008 may be encapsulated within liposomes or may form complexes thereto, in particular to cationic liposomes. Alternatively, the compositions comprising CG57008 may be complexed to lipids, in particular to cationic lipids. For non-sprayable topical dosage forms, viscous to semi-solid or solid forms comprising a carrier or one or more excipients compatible with topical application and having a dynamic viscosity preferably greater than water are typically employed. Other suitable topical dosage forms include sprayable aerosol preparations wherein the active ingredient, preferably in combination with a solid or liquid inert carrier, is packaged in a mixture with a pressurized volatile (e.g., a gaseous propellant, such as Freon or hydrofluorocarbons) or in a squeeze bottle. Moisturizers or humectants can also be added to pharmaceutical compositions and dosage forms if desired. Examples of such additional ingredients are well-known in the art.

A composition comprising CG57008 can be formulated in an aerosol form, spray, mist or in the form of drops or powder if intranasal administration is preferred. In particular, a composition comprising CG57008 can be conveniently delivered in the form of an aerosol spray presentation from pressurized packs or a nebulizer, with the use of a suitable propellant (e.g., dichlorodifluoromethane, trichlorofluoromethane, dichlorotetrafluoroethane, other hydrofluorocarbons, carbon dioxide or other suitable gas). In the case of a pressurized aerosol the dosage unit may be determined by providing a valve to deliver a metered amount. Microcapsules (composed of, e.g., polymerized surface) for use in an inhaler or insufflator may be formulated containing a powder mix of the compound and a suitable powder base such as disaccharides or starch.

One or more CG57008 proteins may also be formulated into a microcapsule with one or more polymers (e.g., hydroxyethyl starch) form the surface of the microcapsule. Such formulations have benefits such as slow-release.

A composition comprising CG57008 can be formulated in the form of powders, granules, microparticulates, nanoparticulates, suspensions or solutions in water or non-aqueous media, capsules, gel capsules, sachets, tablets or minitablets if oral administration is preferred. Thickeners, flavoring agents, diluents, emulsifiers, dispersing aids or binders may be desirable. Tablets or capsules can be prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

In one embodiment, the compositions of the invention are orally administered in conjunction with one or more penetration enhancers, e.g., alcohols, surfactants and chelators. Preferred surfactants include, but are not limited to, fatty acids and esters or salts thereof, bile acids and salts thereof. In some embodiments, combinations of penetration enhancers are used, e.g., alcohols, fatty acids/salts in combination with bile acids/salts. In a specific embodiment, sodium salt of lauric acid, capric acid is used in combination with UDCA. Further penetration enhancers include, but are not limited to, polyoxyethylene-9-lauryl ether, polyoxyethylene-20-cetyl ether. Compositions of the invention may be delivered orally in granular form including, but is not limited to, sprayed dried particles, or complexed to form micro or nanoparticles. Complexing agents that can be used for complexing with the compositions of the invention include, but are not limited to, poly-amino acids, polyimines, polyacrylates, polyalkylacrylates, polyoxethanes, polyalkylcyanoacrylates, cationized gelatins, albumins, acrylates, polyethyleneglycols (PEG), DEAE-derivatized polyimines, pollulans, celluloses, and starches. Particularly preferred complexing agents include, but are not limited to, chitosan, N-trimethylchitosan, poly-L-lysine, polyhistidine, polyornithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG).

A composition comprising CG57008 can be delivered to a subject by pulmonary administration, e.g., by use of an inhaler or nebulizer, of a composition formulated with an aerosolizing agent.

In a preferred embodiment, a composition comprising CG57008 is formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use.

In another embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as benzyl alcohol or lidocaine to ease pain at the site of the injection. Generally, the ingredients are supplied either separately or mixed together in unit dosage form, for example, as a dry lyophilized powder or water free concentrate in a sealed container, such as a vial, ampoule or sachette, indicating the quantity of active agent. Where the composition is to be administered by infusion, it can be dispensed with an infusion container containing sterile pharmaceutical grade water or saline. Where the composition is administered by injection, an ampoule or vial of sterile water for injection or saline can be provided so that the ingredients may be mixed prior to administration.

A composition comprising CG57008 can be formulated as neutral or salt forms. Pharmaceutically acceptable salts include, but are not limited to, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

In addition to the formulations described previously, a composition comprising CG57008 may also be formulated as a depot preparation. Such long acting formulations may be administered by implantation (for example, subcutaneously or intramuscularly) or by intramuscular injection. Thus, for example, the compositions may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

In one embodiment, the ingredients of the compositions used in accordance to the methods of the invention are derived from a subject that is the same species origin or species reactivity as recipient of such compositions.

The invention also provides kits for carrying out the therapeutic regimens of the invention. Such kits comprise in one or more containers prophylactically or therapeutically effective amounts of the composition of the invention (e.g., a composition comprising one or more CG57008 proteins) in pharmaceutically acceptable form. The composition in a vial of a kit of the invention may be in the form of a pharmaceutically acceptable solution, e.g., in combination with sterile saline, dextrose solution, or buffered solution, or other pharmaceutically acceptable sterile fluid. Alternatively, the composition may be lyophilized or desiccated; in this instance, the kit optionally further comprises in a container a pharmaceutically acceptable solution (e.g., saline, dextrose solution, etc.), preferably sterile, to reconstitute the composition to form a solution for injection purposes.

In another embodiment, a kit of the invention further comprises a needle or syringe, preferably packaged in sterile form, for injecting the formulation, and/or a packaged alcohol pad. Instructions are optionally included for administration of the formulations of the invention by a clinician or by the patient.

In some embodiments, the present invention provides kits comprising a plurality of containers each comprising a pharmaceutical formulation or composition comprising a dose of the composition of the invention (e.g., a composition comprising one or more CG57008 proteins) sufficient for a single administration.

As with any pharmaceutical product, the packaging material and container are designed to protect the stability of the product during storage and shipment. In one embodiment, compositions of the invention are stored in containers with biocompatible detergents, including but not limited to, lecithin, taurocholic acid, and cholesterol; or with other proteins, including but not limited to, gamma globulins and serum albumins. Further, the products of the invention include instructions for use or other informational material that advise the physician, technician, or patient on how to appropriately prevent or treat the disease or disorder in question.

### EXAMPLES

### Example 1

### Expression and Purification of Recombinant CG57008 Polypeptides

The recombinant CG57008 proteins were produced in transfected cell lines and purified by methods known in the art. For example, the extracellular domain, extracellular domain-FC and Ig domain-FC fused CG57008 polypeptides were produced by HEK293T cells stably transfected with isolated nucleotides encoding them (SEQ ID Nos 23, 27 and 29). The polypeptides may include His or V5 tags or the like to aid in purification and detection. The FC fused proteins were purified from conditioned media with protein A sepharose. The extracellular domain conditioned medium was loaded onto a Ni²⁺ affinity column (Qiagen, Valencia, Calif.). The column was washed with PBS (pH 7.4), containing 500 mM NaCl, followed by the same buffer containing 5 mM imidazole. The bound protein was eluted with PBS (pH 7.4), containing 500 mM imidazole, pooled, and dialyzed overnight in PBS (pH 7.4). The protein was further purified by a second round of purification over a Ni²⁺ affinity column and dialyzed against PBS (pH 7.4). Protein concentrations were determined using the Bradford reagent (Bio-Rad, Hercules, Calif.). Molarity was calculated using the molecular weight of the dimer. Protein purity was assessed by Silver staining after SDS-PAGE analysis using a 4-15% Tris/glycine gradient gel. Western blot analysis was performed with anti-V5 tag mAb (1:5000; Ausubel et al., eds., CURRENT PROTOCOLS IN MOLECULAR BIOLOGY, Greene Publishing Associates and Wiley-Interscience, New York, 1994) conjugated to horseradish peroxidase, followed by enhanced chemiluminescence (Amersham Pharmacia Biotech, Piscataway, N.J.).

### Example 2

### CG57008 Inhibits T Cell Receptor (TCR)-mediated Responses Methods and Results

Purified CD4+ T cells from healthy blood donors were activated with anti-CD3, and treated with CG57008 at 40 or 125 nM, or negative control protein at 40 or 125 nM, immobilized on culture plates, for 72 h. T cell proliferation was measured in triplicate by BrdU incorporation. BrdU (10 μM final concentration) was added and incubated with the cells for 5 h. BrdU incorporation was assayed according to the manufacturer's specifications (Boehringer Mannheim, Indianapolis, Ind.).

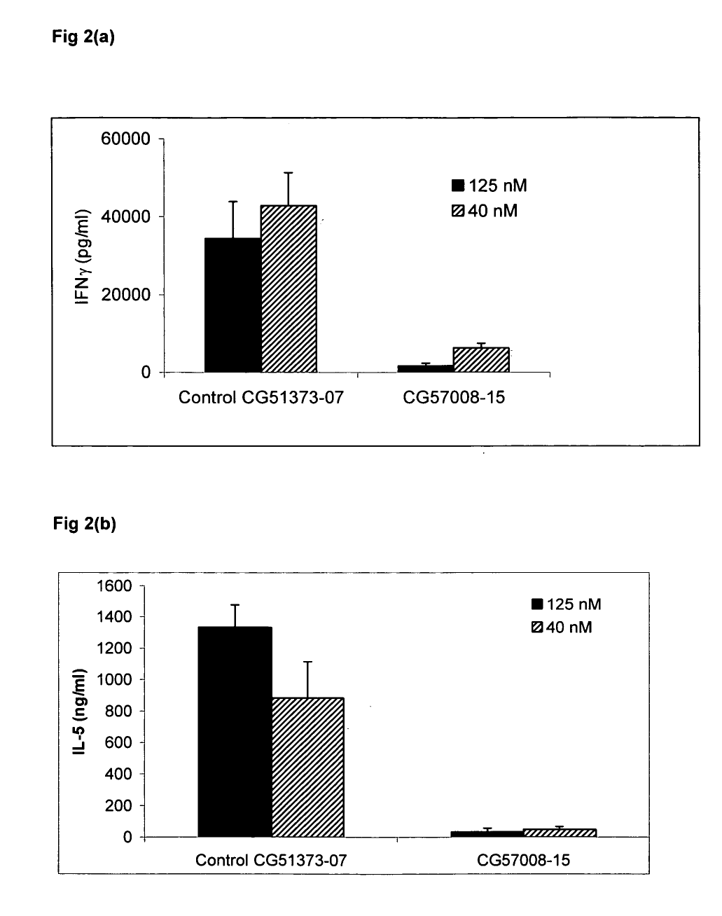

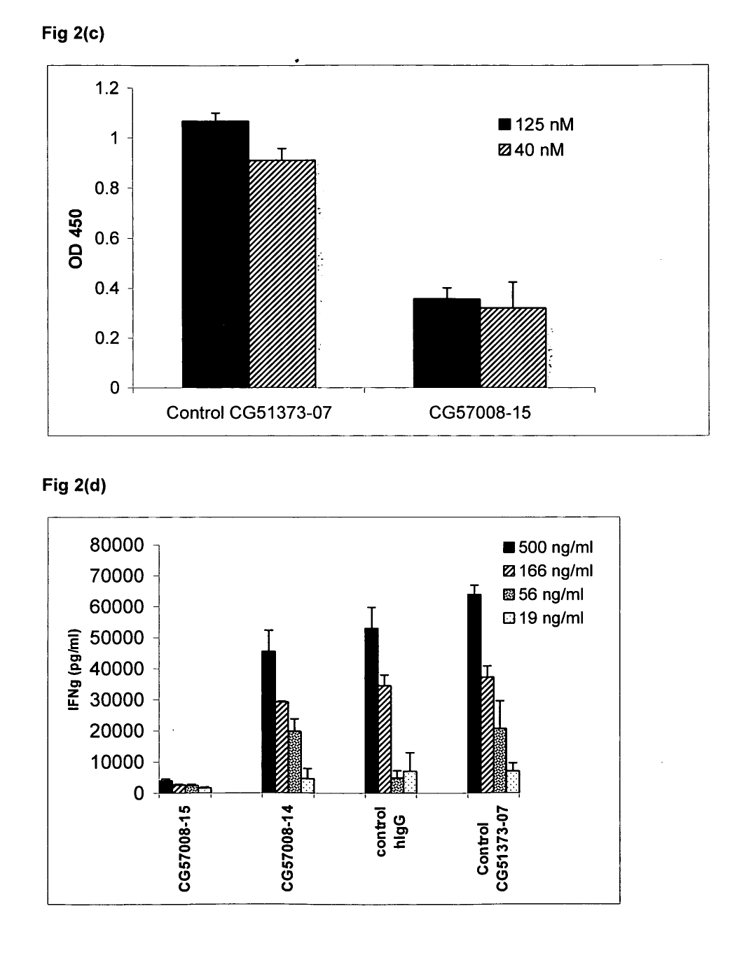

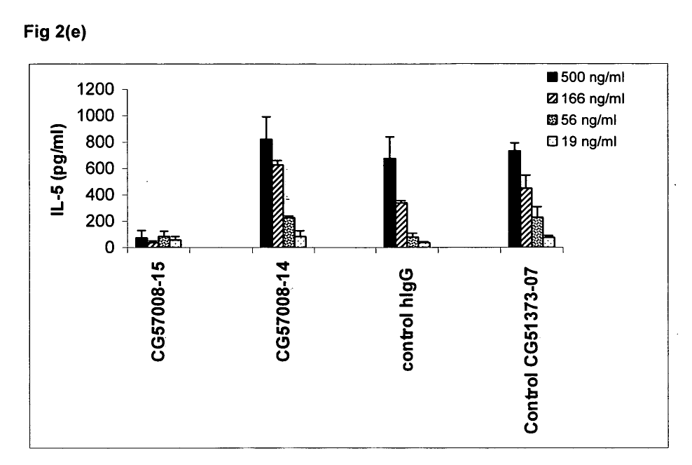

As shown in FIG. 2c, T cell proliferation was markedly reduced as a result of treatment with CG57008-15 at either 40 or 125 nM, as compared to control protein at the same concentrations. T cell release of cytokines was measured by ELISA. T cells activated with immobilized anti-CD3 and treated with CG57008-15 at either 40 or 125 nM concentrations showed a marked decrease in release of IFN-gamma and Interleukin 5 (IL-5) relative to anti-CD3 activated cells treated with control protein at the same concentrations (FIG. 2a-b).

CD4+ T cells were activated with increasing concentrations of immobilized anti-CD3 (19, 56, 166, or 500 ng/ml) and treated with CG57008-15 or a control protein for 72 h. Cytokine release was measured by ELISA. The results showed a significant inhibition of IFN-gamma and IL-5 production by T cells treated with CG57008-15 relative to control protein. (FIG. 2d-e).

### Example 3

### CG57008 Inhibits T Cell Receptor (TCR)-CD28 co-Stimulatory responses

Optimal T cell clonal expansion requires both TCR and CD28 signals. In this experiment, the inventors investigated whether co-stimulatory signals could be blocked by CG57008 polypeptides.

For preparation of Th2 cells, 6 well non tissue culture treated plate was immobilized with anti-CD3 (2 ug/ml) and anti-CD28 (10 ug/ml) (600 ul total in Dulbecco's PBS) overnight at 4° C. The plate was washed with PBS and CD4 lymphocytes were suspended at 500,000 cells/ml in Th2 medium:DMEM+10% FCS+supplements+IL-2 5 ng/ml, IL-4 5 ng/ml, anti-IFN gamma 5 ug/ml and stimulated 4-6 days in CO2 incubator. Then wash 2× in DMEM and rest for 4-6 days in DMEM, 10% FCS+supplements+2 ng/ml IL-2 (500,000 cells/ml). The process of activation and resting is repeated at least once more with the addition of anti-CD95L to prevent apoptosis of cells through FAS.

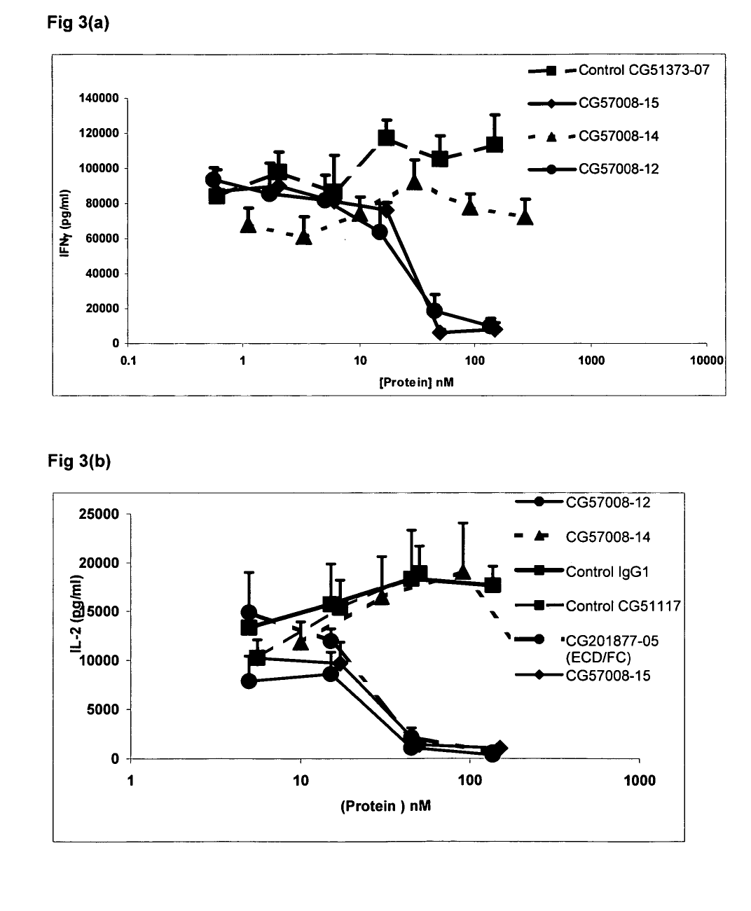

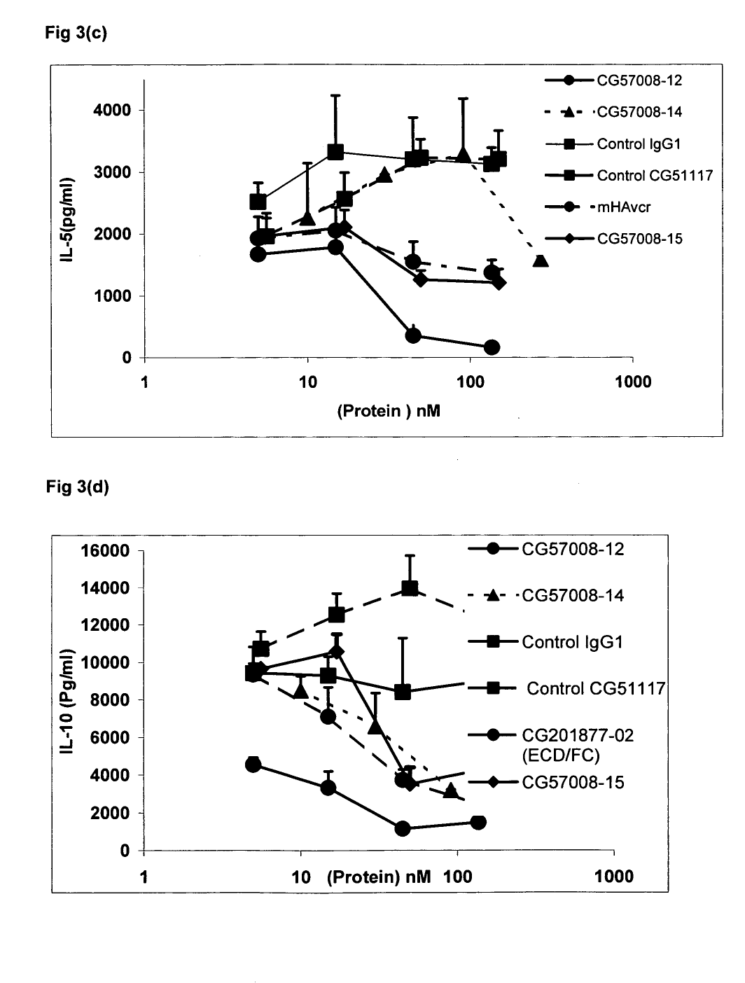

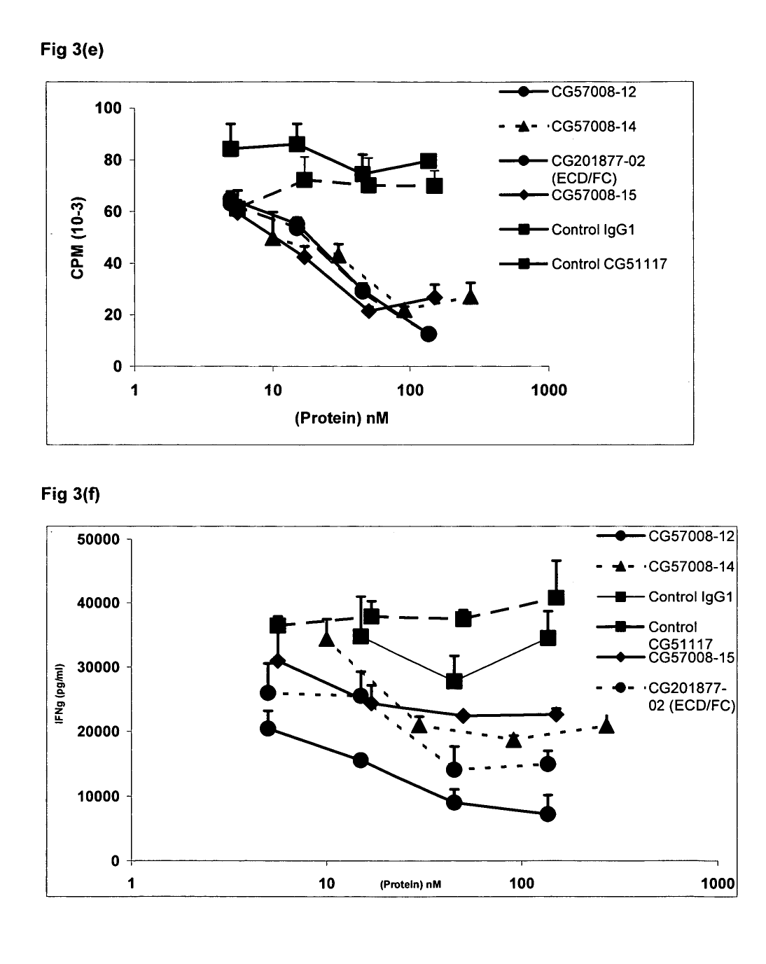

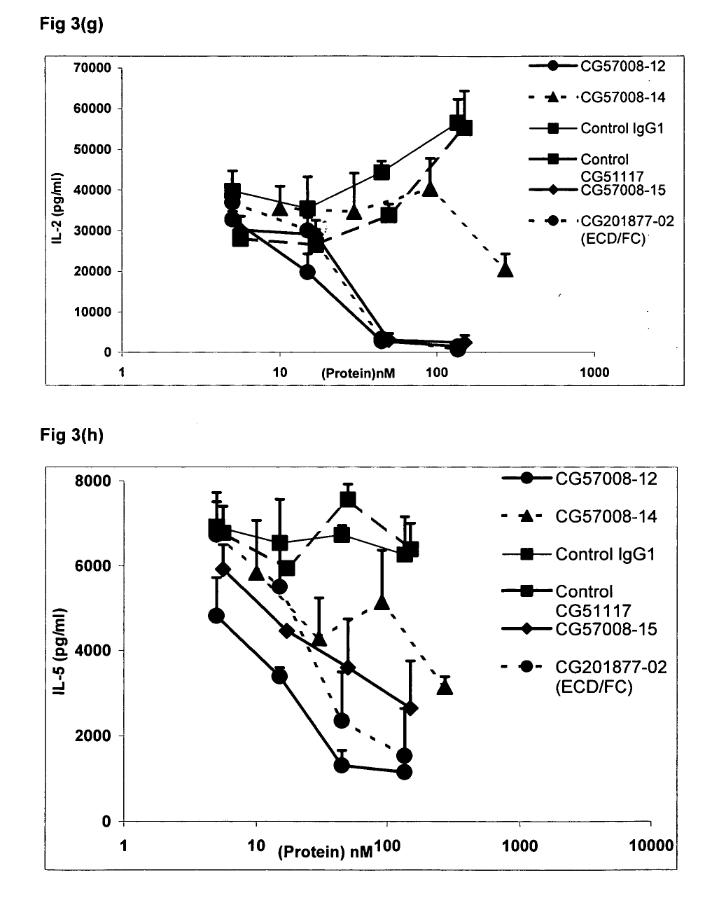

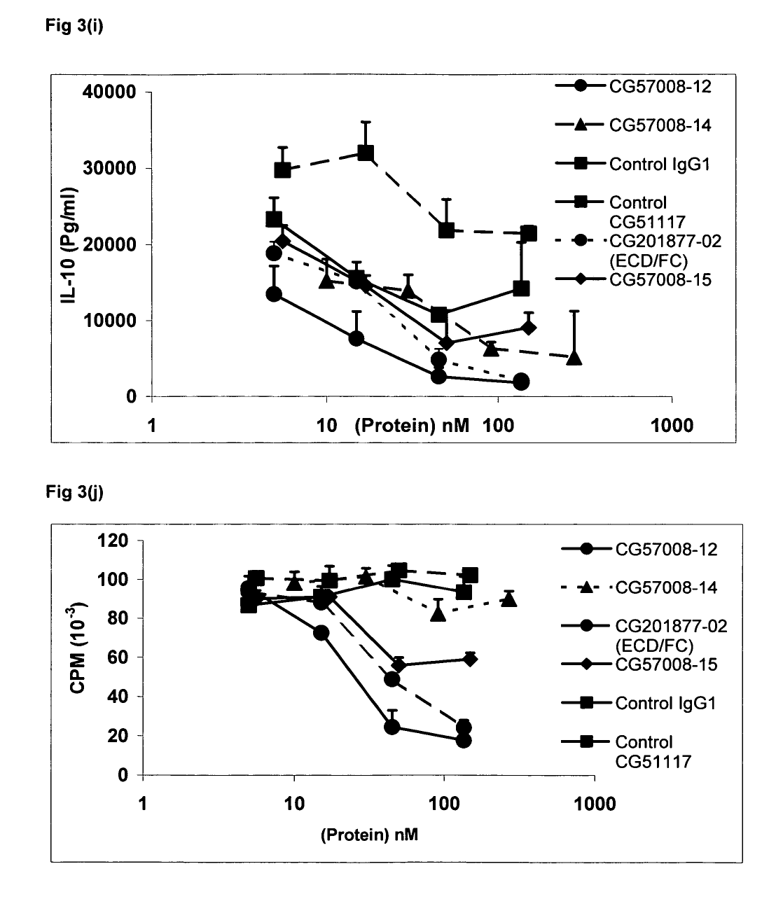

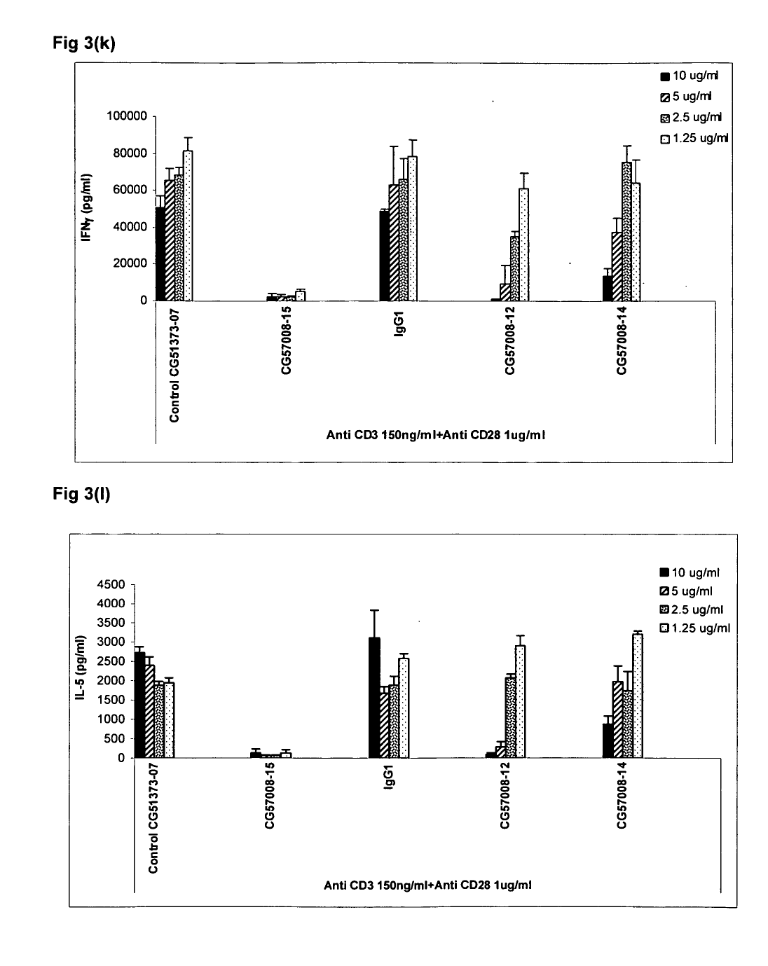

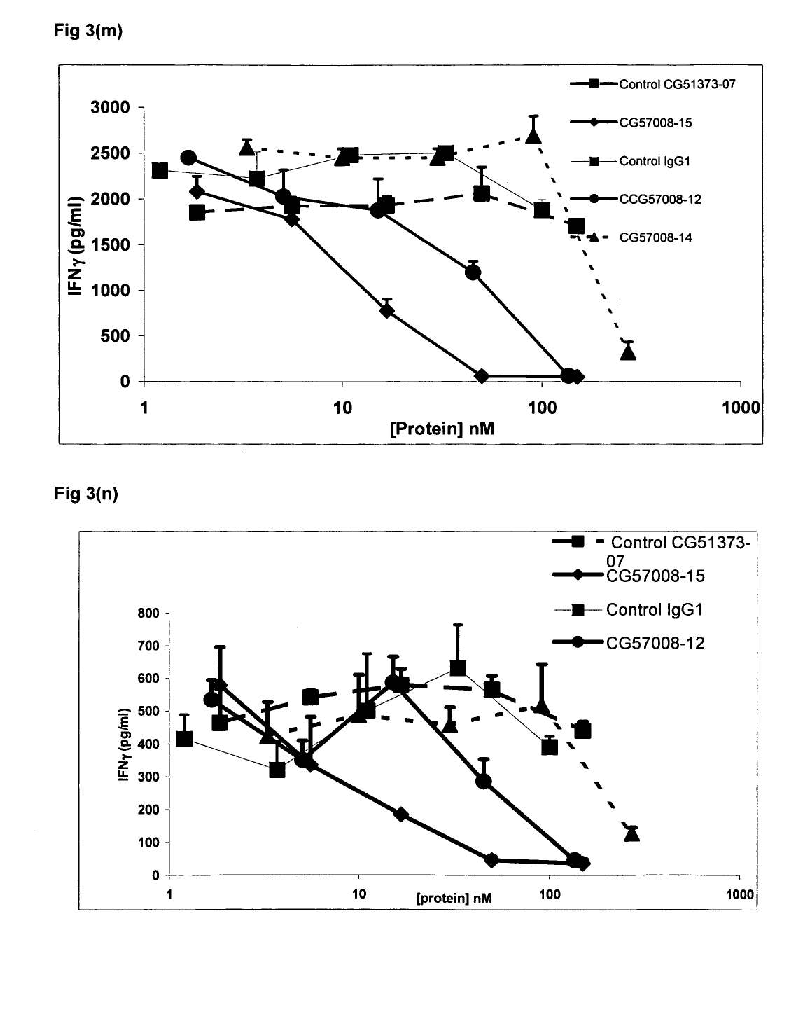

96-well plates were coated with the anti-CD3 and anti-CD28 mAb at 150 ng/ml and 1 μg/ml respectively in PBS overnight at 4° C., aspirated and coated with the control protein, CG57008-15, CG57008-12 or CG57008-14 for 4 hr at 37° C. Wells were aspirated and 150 μl of purified Human CD4+, CD8+ or polarized T_{H}2 cells from healthy blood donors, or purified CD4+ or CD8+ T cells from B6 mice at a concentration of 0.7×10⁶ cells/ml were added and cultured for 72 h at 37° C. Proliferation was measured by [³H] thymidine incorporation by pulsing cells with 0.5 μCi of [³H] thymidine 8 hr prior to harvesting. Cytokines were measured by ELISA using cells harvested at 72 hr. Treatment with CG57008-12, CG57008-15, CG57008-14 and CG201877-02 (ECD/FC) significantly inhibited proliferation of CD8 T cells (FIG. 3e). Treatment with CG57008-12, CG57008-15, and CG201877-02 (ECD/FC) significantly inhibited proliferation of CD4 T cells (FIG. 3j).

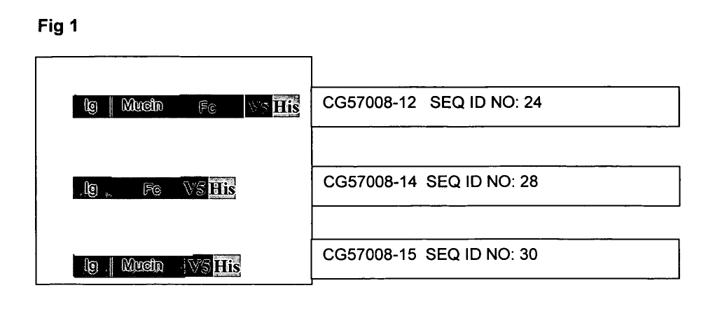

The inventors further demonstrated the inhibition of the secretion of IL-2, IL-5, IL-10 and IFN-γ from anti-CD3, anti-CD28 costimulated T cells by CG57008-15 or CG57008-12 (FIG. 3a-d and 3f-i). CG57008-15 or CG57008-12 also significantly inhibited IFNgamma and IL-5 release from polarized T_{H}2 cells in a concentration dependent manner (FIG. 3k-1). The results clearly demonstrate that CG57008 treatment results in inhibition of TCR-CD28 mediated T cell proliferation and pro-inflammatory cytokine production.

### Example 4

### CG57008 Inhibits Swelling Response in the Oxazalone-Induced Model of Contact Hypersensitivity in Mice

As shown in examples 2 and 3 above, CG57008-15 and CG57008-05 inhibited the transition of naïve T cells into effector cells in vitro. The effect of CG57008 on the sensitization phase of contact hypersensitivity (CHS), a form of delayed-type hypersensitivity (R. Coico editor. Current Protocols in Immunology Online. John Wiley & Sons 2003) was investigated. In this model, during the sensitization phase animals are painted on the abdomen with oxazalone, a hapten, which modifies self-proteins such that they become immunogenic. Langerhans cells and skin dendritic cells move to the local draining lymph node and present the antigen to naïve T cells (J Meingassner et al. 2003, Journal of Investigative Dermatology 121(1), 77). This is the critical time point since CG57008 regulates naïve T cell activation and proliferation in vitro. In the challenge phase of the CHS response, animals are once again treated with oxazalone, but this time on the ear. The second treatment elicits a recall response, attracting the activated effector cells generated in the sensitization phase to the ear, but not naïve T cells. Effector T cells produce cytokines and chemokines and regulate the type and duration of the inflammatory response. The ear swelling response in BALB/c mice is maximal at 24-48 hours and is a result of a massive influx of leukocytes and edema, which resolves in 4-5 days. This response is evaluated by measuring ear thickness with calipers. Baseline thickness is measured on the both ears before treatment. One ear is treated with hapten and the other ear with diluent alone. Ear swelling is the thickness of the treated ear minus the thickness of the diluent treated ear (ΔT). Histological analyses of the ears after treatment were used to verify that changes in swelling correspond to an inflammatory cell influx and not edema alone.

Methods

Animals (10/group), housed 4-5/cage, were acclimated for 4-8 days. Intraperitoneal (IP) dosing with various proteins and controls was initiated on day −1 before sensitization and continued on day 0 and day +2 (see Table 4). For sensitization, mice were anesthetized with isoflourane, and the abdomen shaved and painted with 150 μl oxazalone. Prior to challenge on day 6, the ear thickness was measured to establish baseline thickness. For challenge, animals were anesthetized and treated with 20 μl oxazalone on the left ear. The right ear was treated with ethanol:acetone diluent. Oxazalone (Sigma) was prepared by making a 5% solution in 3:1 ethanol:acetone diluent for sensitization and a 3% oxazalone solution in the same diluent for challenge. Peak inflammation was at 1-2 days after challenge.

The following Table 4 provides a summary of the study design.

TABLE 4
Study Design DTH

| | | | IP Dose | |
|---|---|---|---|---|
| Group | | Number of | ml/kg per day @ | |
| number | Treatment | Mice | day −1, day 0, day 2 | DOSAGE |
| 1 | PBS | 10 | 0 | |
| 2 | IgG1 | 10 | 5 | 5 ml/kg |
| 3 | Cyclosporine | 10 | 10 | 10 ml/kg |
| 4 | CG57008-05 | 10 | 5 | 5 ml/kg |
| 5 | CG57008-15 | 10 | 5 | 5 ml/kg |

Both ears were measured on every other day starting on day 7 until day 21, at the termination of the study. Three animals from each group were sacrificed at day 7 after ear measurement. In addition, ears from each animal were removed, a 7 mm core prepared, weighed, fixed in formalin, embedded in paraffin, stained with hematoxylin-and-eosin (H&E) and used for histological analysis. After final measurements on day 21, all animals were euthanized, three animals from selected groups were used for histological analysis as described above. For the remainder of the animals, 7 mm sections of both ears were weighed, fixed and embedded in paraffin.

Results

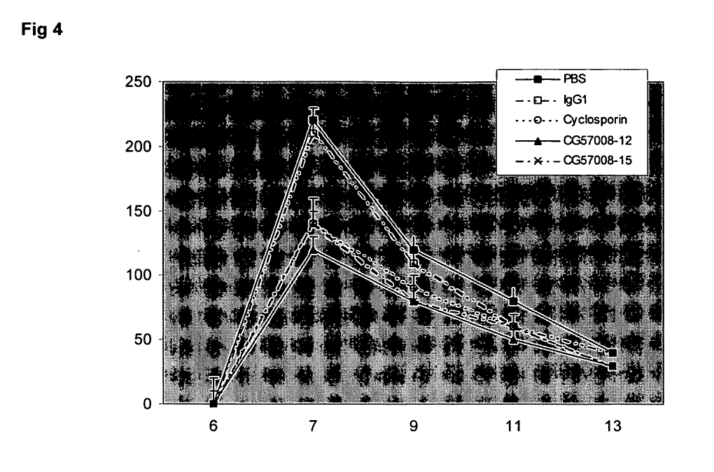

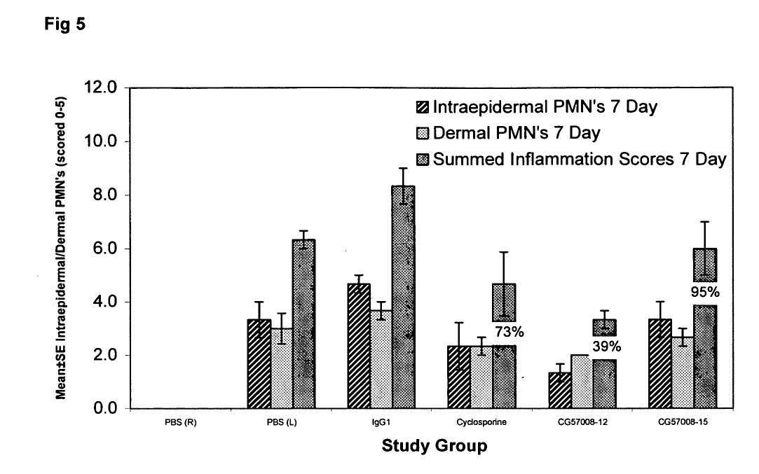

The results of this experiment (FIG. 4) show that challenge with oxazolone caused a significant increase in ear swelling (paired T test comparing day 6 before challenge and day 7 after challenge P<0.0001). Two forms of CG57008 reduced the ear swelling response as compared to appropriate controls (change in ear swelling response. In the group treated with CG57008-05, ear thickness was 124+/−11 μm, while ear thickness in the IgG1 treated control group was 207+/−16 μm (p<0.001). In the group treated with CG57008-15, ear thickness was 136+/−14 μm, while ear thickness in the PBS treated control group was 218+/−13 μm (p<0.0013; based on one-way ANOVA). Cyclosporine, an immunosuppressant drug that significantly reduces T cell responses and ear swelling in models of contact hypersensitivity, was used as a positive control in this experiment. Results obtained with CG57008 were equivalent to results seen in animals treated with cyclosporine, which reduced ear swelling 140+/−10 μm (p<0.0006 as compared to PBS). Although the greatest change in ear swelling was found at 24 hours after challenge, there were also statistically significant differences in the overall profile of the response for CG57008-05 over the whole time period measured (p<0.04) as compared to IgG1 and for CG57008-15 as compared to PBS until day 15 of the study (p<0.0001). Furthermore, the CG57008-05 mediated reduction in ear swelling response was correlated to the level of leukocyte infiltration and edema based on histopathological analysis of hematoxylin and eosin stained ear sections from mice sacrificed twenty-four hours after challenge (FIG. 5).

Together these data show that CG57008 (5 mg/kg) inhibits the ear swelling response as well as or better than cyclosporine (10 mg/kg), and shows that CG57008 is a potent immunosuppressant.

### Example 5

### CG57008 Inhibits Inflammation and Joint Damage due to Antigen Induced Arthritis

Antigen induced arthritis (AIA) is a T cell-mediated delayed type hypersensitivity (DTH)-like response localized to the joint that results in the cartilage loss, pannus formation (synovial tissue hyperproliferation), and bone destruction characteristic of human rheumatoid arthritis. This model also has the additional benefit of having a well-defined, consistent and rapid course of disease that distinguishes it from systemic arthrides such as Collagen-Induced Arthritis (CIA) and genetic models. It has been recently used to define the role of TSG-6 (Arthritis Rheum. 2002 August;46(8):2207-18) a protease inhibitory protein the blocks the degradation of cartilage.

Antigen induced foot pad inflammation is also a T cell mediated DTH-like response that results in localized soft tissue swelling in a location (hind paws) that is amenable to sequential caliper measurements and can be used to determine the capacity of potential inhibitory agents to suppress the immune response. Here we examined the ability of CG57008 to inhibit both inflammatory responses in each animal.

We have found that CG57008 blocks T cell activation in vitro and inflammation in T cell mediated contact hypersensitivity in vivo suggesting that CG57008 modulates the function of activated T cells. We wanted to confirm that CG57008 could block inflammation in soft tissues (i.e. the foot pad) and determine if CG57008 could also block joint destruction in a model of T cell mediated arthritis. Inflammation was induced in the footpad by intramuscular injection of methylated bovine serum albumin (mBSA) and arthritis was induced in the knee joints by intra-articular injection of mBSA. Footpad inflammation was measured by evaluating foot swelling (caliper measurements of left vs. right hind paw) and by histological assessment of H&E stained slides. Joint destruction was assessed by scoring histopathologic inflammation, pannus formation, cartilage damage and bone resorption. In each mouse, both antigen induced arthritis and foot pad inflammation were measured.

Methods

Mice were rested for 8 days, weighed and randomized into groups on day −(1) as described in Table 5. Animals in groups 8, 10, 11, 12 were dosed ip, qd on days (−)1, 0, 2 and 6, 7, 9 around the time of mBSA sensitization. Animals in groups 2-3 & 5, 6, 7 were dosed ip, qd from days 13-18 around mBSA challenge. Group 9 was dosed po, qd on days (−)1-9 with Dexamethasone (0.1 mg/kg). Animals in group 4 were dosed po, qd on days 13-18 with Dexamethasone (0.2 and 0.3 mg/kg).

TABLE 5
Study Design AIA

| | Disease induction | Number of | | | |
|---|---|---|---|---|---|
| Group | (arthritic) | mice | Treatment | Schedule | Dosage |
| 1 | No | 3 | None | N/A | N/A |
| 2 | Yes | 10 | PBS | Once daily d | N/A |
| | | | | 13-18 | |
| 3 | Yes | 10 | IgG1 | Once daily d | 15 mg/kg ip |
| | | | | 13-18 | |
| 4 | Yes | 10 | Dexamethasone | Once daily d | 0.2 (d13) |
| | | | | 13-18 | and 0.3 mg/kg |
| | | | | | (d14-18), |
| | | | | | po |
| 5 | Yes | 10 | CG57008-05 | Once daily d | 15 mg/kg ip |
| | | | | 13-18 | |
| 6 | Yes | 10 | CG57008-05 | Once daily d | 5 mg/kg ip |
| | | | | 13-18 | |
| 7 | Yes | 10 | CG57008-05 | Once daily d | 1 mg/kg ip |
| | | | | 13-18 | |
| 8 | Yes | 10 | IgG1 | Treat once | 15 mg/kg ip |
| | | | | daily on days | |
| | | | | −1, 0, 2 and | |
| | | | | days 6, 7, 9 | |
| 9 | Yes | 10 | Dexamethasone | Days (—) 1-9 | 0.1 mg/kg, |
| | | | | | po |
| 10 | Yes | 10 | CG57008-05 | Treat once | 15 mg/kg ip |
| | | | | daily on days | |
| | | | | −1, 0, 2 and | |
| | | | | days 6, 7, 9 | |
| 11 | Yes | 10 | CG57008-05 | Treat once | 5 mg/kg ip |
| | | | | daily on days | |
| | | | | −1, 0, 2 and | |
| | | | | days 6, 7, 9 | |
| 12 | Yes | 10 | CG57008-05 | Treat once | 1 mg/kg ip |
| | | | | daily on days | |
| | | | | −1, 0, 2 and | |
| | | | | days 6, 7, 9 | |

For the sensitization phase of disease induction, animals in groups 2-12 were anesthetized with isoflurane and given intradermal injections of antigen with adjuvant (0.1 ml) at the base of the tail on days 0 and 7. Antigen was prepared by combining an equal volume of mBSA (4 mg/ml in sterile water) with Freund's complete adjuvant containing 4 mg/ml M. tuberculosis. The mixture was sonicated for 5 minutes and emulsified by hand mixing for approximately 5 minutes before injection.

On day 14, all animals (including group 1 non-sensitized) were challenged by injecting antigen into the right knee joint (10 μl of 10 mg/ml mBSA in water) and footpad (20 μl of 10 mg/ml mBSA). To measure DTH in the foot over time, right and left ankles (medial to lateral) of the animals were measured every 24 hours for five days (day 15-19). Five days later (day 19), mice from all the groups were sacrificed, serum collected and knee and paw tissues harvested. All tissues were fixed in formalin, stained and scored as described below. Evaluation of arthritis in the knee and inflammation were assessed by histological analysis.

Processing of Joints

Following 1-2 days in fixative and then 4-5 days in decalcifier, the knees were processed, embedded in the frontal plane, sectioned and stained with toluidine blue. Ankles were embedded in the sagittal plane and stained with hematoxylin and eosin.

Scoring of Knee Joints knees were given scores of 0-5 for inflammation, pannus formation, cartilage damage and bone resorption according to the following criteria:

Inflammation
0=Normal
1=Minimal infiltration of inflammatory cells in periarticular tissue
2=Mild infiltration
3=Moderate infiltration with moderate edema
4=Marked infiltration with marked edema
5=Severe infiltration with severe edema

Pannus
0=Normal
1=Minimal infiltration of pannus in cartilage and subchondral bone
2=Mild infiltration (<¼ of tibia or femur affected at edges)
3=Moderate infiltration (¼ to ⅓ of tibia or femur affected)
4=Marked infiltration (½-¾ of tibia or femur affected)
5=Severe infiltration (>¾ of tibia or femur affected), severe distortion of overall architecture

Cartilage Damage
0=Normal
1=Minimal, minimal to mild loss of toluidine blue staining with no obvious chondrocyte loss or collagen disruption
2=Mild, mild to moderate (full thickness) loss of toluidine blue staining with or without focal minimal (superficial) chondrocyte loss and/or collagen disruption
3=Moderate, moderate loss of toluidine blue staining with multifocal moderate (depth to middle zone) chondrocyte loss and/or collagen disruption
4=Marked, marked loss of toluidine blue staining with multifocal marked (depth to deep zone) chondrocyte loss and/or collagen disruption
5=Severe, severe diffuse loss of toluidine blue staining with multifocal severe (depth to tide mark or greater) chondrocyte loss and/or collagen disruption

Bone Resorption
0=Normal
1=Minimal, small areas of resorption at joint margins, not readily apparent on low magnification
2=Mild, more numerous areas of resorption, not readily apparent on low magnification, <¼ of tibia or femur at edges resorbed
3=Moderate, obvious resorption of medullary trabecular and cortical bone, lesion apparent on low magnification, ¼ to ⅓ of tibia or femur affected
4=Marked, full thickness defects in cortical bone, often with distortion of profile of remaining cortical surface, marked loss of medullary bone, ½-¾ of tibia or femur affected
5=Severe, full thickness defects in cortical bone of tibia and or femur, often with distortion of profile of remaining cortical surface, marked loss of medullary bone, >¾ of tibia or femur affected, severe distortion of overall architecture

Scoring of Ankle Joints ankles are given scores of 0-5 for inflammation according to the following criteria:
0=normal
1=minimal infiltrate
2=mild infiltrate and edema
3=moderate infiltrate and edema
4=marked infiltrate and edema with fibrin exudation and microabscesses
5=severe infiltrate and edema with fibrin exudation and microabscesses
   Results

This experiment was designed to determine if CG57008-05 could reduce soft tissue and joint inflammation and damage in a T cell dependent model of antigen induced arthritis. Sensitization and challenge with mBSA induced swelling starting at 1 day after challenge and continuing to 5 days after challenge. The swelling in sensitized and challenged animals treated with diluent (PBS) or IgG1 was significantly greater than in animals that were only challenged and not sensitized (p<0.05). Showing that both sensitization and challenge are required for the response and that antigen alone is not sufficient to induce inflammation.

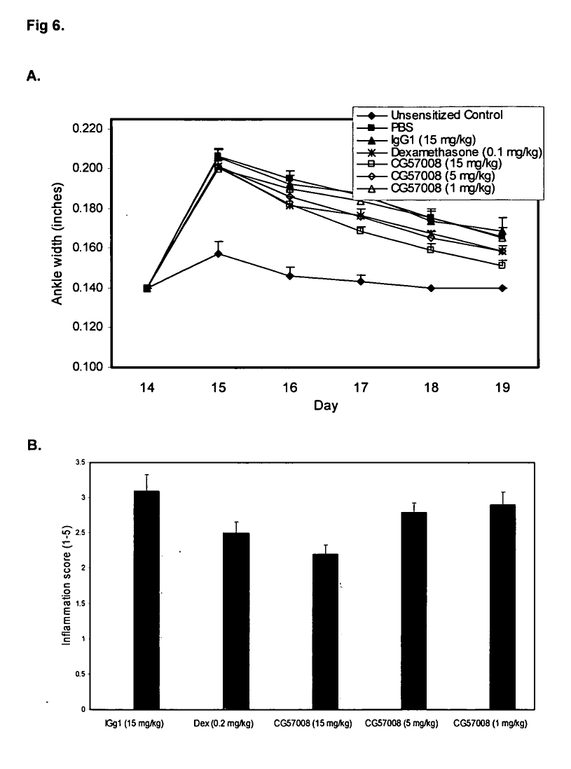

Treatment with CG57008-05 (15 mg/kg) during sensitization phase of disease on days (−)1, 0, 2 and 6, 7, 9 resulted in a significant reduction in ankle swelling (FIG. 6A; p=0.028) and, consistent with the anti-inflammatory effects of CG57008, did not differ from the positive control dexamethasone (p=0.786). The histological analysis of footpad inflammation was also significantly reduced 29% (P=0.007) in animals treated with the same dose of CG57008-15 as compared to negative control group treated with IgG (15 mg/kg; FIG. 6B). Dexamethasone did not significantly reduce this assessment of inflammation.

In contrast, treatment with CG57008-05 at sensitization did not reduce inflammation in the knee, or the subsequent damage in the bone, synovial membrane or cartilage. Dexamethasone also did not significantly reduce knee inflammation but it did slightly, but significantly, reduce pannus and bone damage (p<0.05; data not shown).

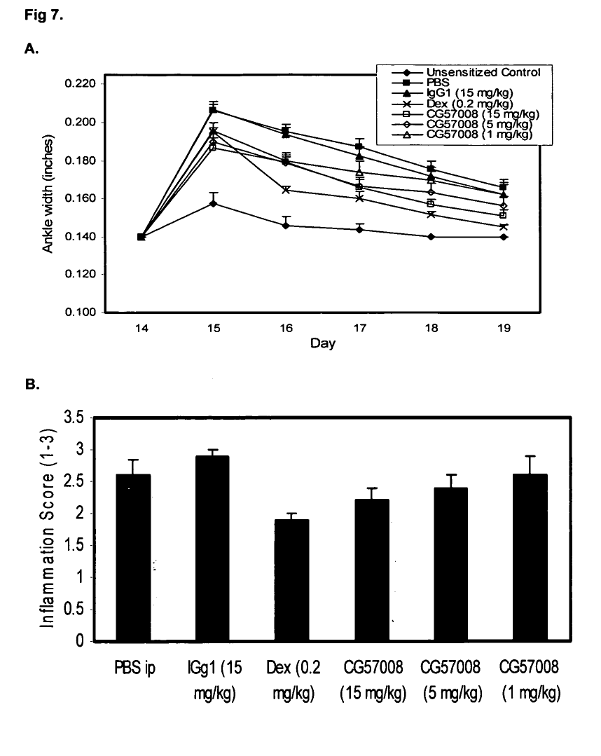

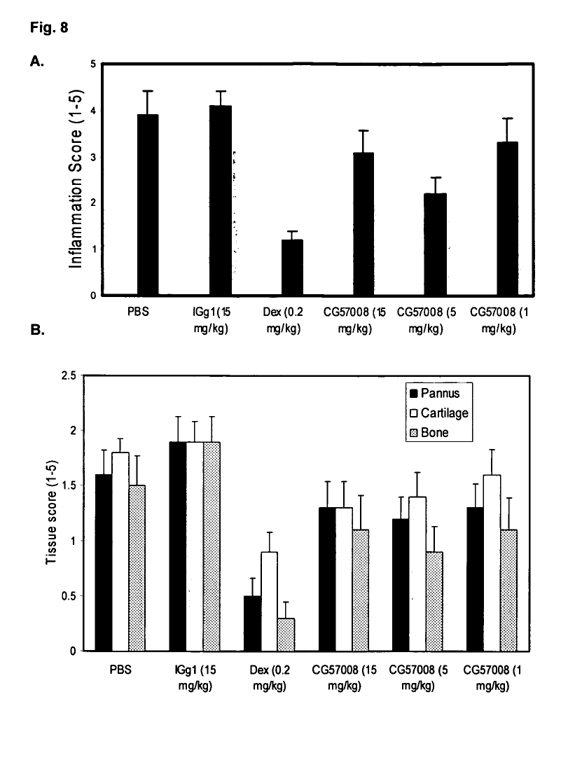

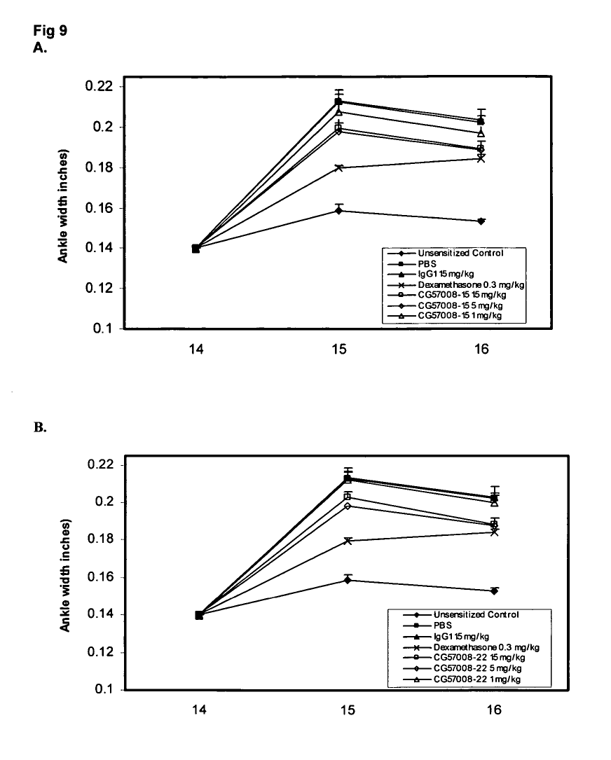

Treatment with CG57008-05 (15 mg/kg) around the time of antigenic challenge (days 13-18) significantly reduced ankle swelling when compared to PBS (p=0.005) and IgG1 (p=0.018), but not dexamethasone (p=0.762) over all time points (FIG. 7A). Furthermore, both the high (15 mg/kg) and middle dose (5 mg/kg) of CG57008-05 had a significant effect three days after challenge (day 17; p<0.05). Histopathological inflammation (FIG. 7B) was also slightly reduced in animals treated with CG57008-05 as compared to controls (5 mg/kg; p=0.03).

CG57008-05 (5 mg/kg) was also able to reduce inflammation in the knee by 46% as compared to IgG (15 mg/kg) treated controls (FIG. 8A; p=0.03). Consistent with the reduction in inflammation, treatment with this dose of CG57008-05 also reduced pannus formation to 63% (p=0.014) and bone resorption to 47% (p=0.010) of IgG treated controls. Dexamethasone treatment also decreased these parameters significantly as well as reducing cartilage damage (FIG. 8B).

Together these studies confirm the results in example 4 and show CG57008-05 inhibits inflammation due to T cell activation. Furthermore, this experiment shows that the efficacy of CG57008-05 is not site (ear vs. footpad or knee) or antigen (oxazalone vs. mBSA/adjuvant) specific and the suppression of T cell activation and/or inflammation is great enough at some doses of CG57008-05 to alter the course of arthritic joint destruction including pannus formation and bone damage. These studies also compared two treatment schemes, treatment when naïve T cells were sensitized (days—1, 0, 2 and 6, 7, 9) and when effector T cells were activated (days 13-18). The results show CG57008-05 given at either time reduced ankle swelling and foot pad inflammation and that the later treatment with CG57008-05 also reduced knee joint damage.

### Example 6

### Fusion of the Fc Portion of Human IgG1 to CG57008 does not Enhance Efficacy Based on a Comparison of CG57008-15 and CG57008-22 in Antigen Induced Paw Swelling

To determine if addition of the Fc portion of human IgG1 was required or enhanced the efficacy of CG57008 to reduce inflammation, we compared the ability of CG57008-15, which does not have an Fc tag, and CG57008-22, which does have an Fc tag, to reduce antigen induced paw/ankle swelling using the same protocol described in Example 5. The only differences in protocol were that mice were only treated with CG57008 or control drugs on days 13-15 around the time of challenge and the animals were sacrificed two days after challenge rather than five days after challenge.

TABLE 6
Study Design comparison of CG57008-05 and CG57008-22

| | Disease | Number | | | |
|---|---|---|---|---|---|
| | induction | of | | | |
| Group | (arthritic) | mice | Treatment | Schedule | Dosage |
| 1 | No | 3 | None | N/A | N/A |
| 2 | Yes | 10 | PBS ip | Once daily | N/A |
| | | | | days 13-15 | |
| 3 | Yes | 10 | IgG1 | Once daily | 15 mg/kg ip |
| | | | | days 13-15 | |
| 4 | Yes | 10 | Dexa- | Once daily | 0.3 mg/kg, |
| | | | methasone | days 13-15 | po |
| 5 | Yes | 10 | CG57008-22 | Once daily | 15 mg/kg ip |
| | | | | days 13-15 | |
| 6 | Yes | 10 | CG57008-22 | Once daily | 5 mg/kg ip |
| | | | | days 13-15 | |
| 7 | Yes | 10 | CG57008-22 | Once daily | 1 mg/kg ip |
| | | | | days 13-15 | |
| 8 | Yes | 10 | CG57008-05 | Once daily | 15 mg/kg ip |
| | | | | days 13-15 | |
| 9 | Yes | 10 | CG57008-05 | Once daily | 5 mg/kg ip |
| | | | | days 13-15 | |
| 10 | Yes | 10 | CG57008-05 | Once daily | 1 mg/kg ip |
| | | | | days 13-15 | |

Results

CG57008 treated animals were compared to IgG treated controls. The change in ankle width was significantly reduced in animals treated with CG57008-05 (FIG. 9A) at doses of 15 mg/kg and 5 mg/kg (both p<0.05). Animals treated with CG57008-22 (FIG. 9B) at 15 mg/kg had reduced inflammation that did not reach significance (p=0.067); however the 5 mg/kg dose did significantly reduce in ankle width (p=0.019). These data indicate that addition of the Fc portion of the human IgG1 protein to CG57008 is not required for the efficacy of CG57008 treatment of acute antigen induced inflammation in the paw.

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims. The claims presented are representative of the inventions disclosed herein. Other, unclaimed inventions are also contemplated. Applicants reserve the right to pursue such inventions in later claims.

1. A method of inhibiting the release of cytokines from immune cells comprising contacting the cells with a composition comprising a CG57008 polypeptide.

2. The method of claim 1, wherein the CG57008 polypeptide is selected from the group consisting of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, and 82.

3. The method of claim 1, wherein the immune cells are T-cells.

4. The method of claim 1, wherein the immune cells are CD4+ T-cells or CD8+ T-cells or T_{H}2 cells.

5. The method of claim 1, wherein the cytokines are IL-2, IL-5, IL-10 or IFN-gamma.

6. A method for inhibiting the proliferation of immune cells, comprising contacting the cell with a composition comprising a CG57008 polypeptide.

7. The method of claim 6 wherein the CG57008 polypeptide is selected from the group consisting of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, and 82.

8. The method of claim 6, wherein the immune cells are T-cells.

9. The method of claim 6, wherein the immune cells are CD4+ T-cells or CD8+ T-cells.

10. A method for treating or preventing a delayed-type contact hypersensitivity in a mammal comprising administration of a composition comprising a CG57008 polypeptide to the mammal in need of such treatment.

11. The method of claim 10 wherein the CG57008 polypeptide is selected from the group consisting of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, and 82.

12. A method for treating or preventing arthritis in a mammal comprising administration of a composition comprising a CG57008 polypeptide to the mammal in need of such treatment.

13. The method of claim 12 wherein the CG57008 polypeptide is selected from the group consisting of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, and 82.

14. A method of suppressing a T-cell mediated immune reaction or inflammatory disorder comprising administration of a composition comprising a CG57008 polypeptide.

15. The method of claim 14 wherein the CG57008 polypeptide is selected from the group consisting of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, and 82.

16. The method of claim 14, wherein the immune reaction or inflammatory disorder is selected from the group consisting of asthma, allergy, atopic eczema, inflammatory bowel disease, systemic lupus erythematosus, rheumatoid arthritis, immune thrombocytopenic purpura, chronic obstructive pulmonary disease, psoriasis, contact hypersensitivity, multiple sclerosis, solid organ transplant, rejection of bone marrow transplant, graft versus host disease, and Sezary's Syndrome.

17. A method of preventing or alleviating a symptom of an immune reaction or inflammatory disorder comprising administration to a subject a composition comprising a CG57008 polypeptide.

18. The method of claim 17 wherein the CG57008 polypeptide is selected from the group consisting of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, and 82.

19. The method of claim 17, where the immune reaction or inflammatory disorder is mediated by T-cells activation.

20. The method of claim 17, wherein the immune reaction or inflammatory disorder is selected from the group consisting of asthma, allergy, atopic eczema, inflammatory bowel disease, systemic lupus erythematosus, rheumatoid arthritis, antigen-induced arthritis, purpura, chronic obstructive pulmonary disease, psoriasis, contact hypersensitivity, multiple sclerosis, solid organ transplant, rejection of bone marrow transplant, graft versus host disease, and Sezary's Syndrome.

21. An isolated polypeptide comprising the mature form of an amino acid sequences selected from the group consisting of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, and 82.

22. An isolated polypeptide comprising an amino acid sequences selected from the group consisting of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, and 82.

23. A composition comprising the polypeptide of claim 21 and a carrier.
